SEQUENCE LISTING (6 sequences)

SEQ ID NO: 1 - protein, 42 aa, Homo sapiens [sequence omitted]

SEQ ID NO: 2 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 3 - protein, 126 aa, Homo sapiens
MDVFMKGLSKAKEGVVAAAEKTKQGVAEAAGKTKEGVLYVVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 4 - protein, 112 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKEGYQDYEPEA

SEQ ID NO: 5 - protein, 22 aa, Homo sapiens
FFAEDVGSNKGAIIGLMVGGVV

SEQ ID NO: 6 - protein, 8 aa, Homo sapiens
GAVVTGVT

(19) United States
(12) Patent Application Publication
(10) Pub. No.: US 2018/0194822 A1
(43) Pub. Date: Jul. 12, 2018

(54) DISRUPTION OF THE INTERACTION BETWEEN AMYLOID BETA PEPTIDE AND DIETARY LIPIDS

(71) Applicant: THE TRUSTEES OF COLUMBIA UNIVERSITY IN THE CITY OF NEW YORK, New York, NY (US)

(72) Inventor: Laura McIntire, Long Island City, NY (US)

(73) Assignee: THE TRUSTEES OF COLUMBIA UNIVERSITY IN THE CITY OF NEW YORK, New York, NY (US)

(21) Appl. No.: 15/910,517

(22) Filed: Mar. 2, 2018

Related U.S. Application Data
(63) Continuation of application No. PCT/US2016/050265, filed on Sep. 2, 2016.
(60) Provisional application No. 62/214,530, filed on Sep. 4, 2015.
(60) Provisional application No. 62/237,247, filed on Oct. 5, 2015.
(60) Provisional application No. 62/299,289, filed on Feb. 24, 2016.
(60) Provisional application No. 62/299,816, filed on Feb. 25, 2016.

(51) Int. Cl. C07K 14/47; C07K 16/28; G01N 33/68; A61P 25/28; A61P 25/16

(52) U.S. Cl. CPC C07K 14/4711; C07K 16/28; A61B 6/037; A61P 25/28; A61P 25/16; G01N 33/6896

(57) ABSTRACT

The present invention relates to methods of treating neurodegenerative disorders associated with Alzheimer's disease (AD), Parkinson's disease (PD) and synucleinopathies, such as dementia with Lewy bodies, Down Syndrome (DS) and associated cognitive disorders, multiple system atrophy, and rare neuroaxonal dystrophies, such as Niemann-Pick type C disease (NPC) and Gaucher's disease comprising administering an inhibitor to disrupt the interaction between Aβ or αS and neuronal lipids. The invention further relates to assays for identifying agents that reduce interaction between Aβ or αS and neuronal lipids. Lastly, the invention relates to methods and compositions for intranasal administration of fatty acids or lipids containing fatty acid acyl chains of dietary lipids for promoting central nervous system health and/or prevention or treatment of neurodegenerative disorders.

### CROSS REFERENCE TO RELATED APPLICATIONS

This application is a continuation application of International Application No. PCT/US2016/050265, filed Sep. 2, 2016, which claims priority to U.S. Provisional Patent Application Ser. No. 62/214,530, filed Sep. 4, 2015, to U.S. Provisional Patent Application Ser. No. 62/237,247, filed Oct. 5, 2015, to U.S. Provisional Patent Application Ser. No. 62/299,289, filed Feb. 24, 2016, and to U.S. Provisional Patent Application Ser. No. 62/299,816, filed Feb. 25, 2016, the contents of each of which are incorporated by reference in their entireties herein, and priority to each of which is claimed.

### GRANT INFORMATION

This invention was made with government support under Grant Nos. 1R2INS084328-01A1 and 1K01AG047954-01 awarded by the National Institutes of Health. The government has certain rights in the invention.

### SEQUENCE LISTING

The specification further incorporates by reference the Sequence Listing submitted herewith via EFS on Feb. 28, 2018. Pursuant to 37 C.F.R. § 1.52(e)(5), the Sequence Listing text file, identified as “070050_6073_ST25.txt,” is 4,752 bytes and was created on Mar. 1, 2018. The Sequence Listing, electronically filed herewith, does not extend beyond the scope of the specification and thus does not contain new matter.

### 1. INTRODUCTION

The present invention relates to methods of treating neurodegenerative disorders associated with Alzheimer's disease (AD), Down Syndrome (DS) and associated cognitive disorders, Parkinson's disease (PD) and synucleinopathies, such as dementia with Lewy bodies and multiple system atrophy, and rare neuroaxonal dystrophies, such as Niemann-Pick type C disease (NPC) and Gaucher's disease comprising administering an inhibitor to disrupt the interaction between Aβ or αS and neuronal lipids. The invention further relates to assays for identifying agents that reduce the interaction between Aβ or αS and neuronal lipids. In addition, the invention relates to methods and compositions for intranasal administration of fatty acids or lipids containing fatty acid acyl chains of dietary lipids for promoting central nervous system health and/or prevention or treatment of neurodegenerative disorders.

### 2. BACKGROUND OF THE INVENTION

Genetic and pathological evidence have established that accumulation of amyloid β-peptide (Aβ) in the brain is a critical and defining characteristic of AD. Aβ accumulates as soluble oligomers, protofibrils, fibrils and is deposited as plaques in the brain of AD patients as well as animal models (1,2). Much effort in the field to develop therapeutics has been devoted to clearing brain Aβ using passive and active immunotherapies; preventing its accumulation by targeting the synthetic enzymes, gamma and beta secretases directly or by preventing coincidence between secretases and amyloid precursor protein (APP) to prevent cleavage and formation of Aβ (3). However, several failures of late stage clinical trials for these strategies have made it clear that new rationales and therapeutic avenues are required.

Many years of research have established the critical importance of docosahexaenoic acid (DHA; also 22:6) for maintaining normal healthy brain function and vasculature (4-6). Much research has been done in the field of AD implicating DHA and other dietary lipids in prevention or amelioration of AD cognitive decline, although the mechanisms underlying this promising correlation have been elusive. DHA is reduced in red blood cells of AD patients and DHA supplementation abrogates cognitive deficits in several animal models (6,7). Enhanced dietary ingestion of DHA (i.e., the Mediterranean diet) is correlated with reduced risk for developing AD. However, the efficacy of oral DHA supplementation in human clinical trials been reported to be ineffective (6,8). This may be due to inability of the lipophilic DHA to reach the site of action in the brain after administration systemically, usually through oral supplementation.

Aβ is a highly hydrophobic molecule and hydrophobicity increases with the gamma secretase cleavage that produces Aβ42 (hydrophobicity: Aβ42>Aβ40>Aβ38), the peptide correlated with aggregation as well as cellular toxicity (9,10). It is likely that hydrophobicity of Aβ is a critical determinant of its synaptotoxicity, as well as long term chronic toxicity associated with Aβ accumulation in brain (11,12). Further, lipoproteins which bind lipids in the peripheral circulation may sequester and prevent DHA from reaching the brain. Finally, absorption by the gastrointestinal tract and first pass metabolism deter DHA from reaching the brain in sufficient quantities to exert mechanistic actions.

DHA has been used, in non-human animal models, as a lipid carrier for drugs of interest in intranasally administered formulations (93, 100). However, DHA in such formulations has been considered to be relatively inactive, although some anti-inflammatory and cysticidal properties were reported.

There is an unmet need for treatment of AD, DS, PD, synucleinopathies such as dementia with Lewy bodies, multiple system atrophy, and rare neuroaxonal dystrophies, such as NPC and Gaucher's disease, which lead to neurodegeneration. The diseases are characterized by lipid dyshomeostasis, which can putatively hinge on distribution of polyunsaturated fatty acids (PUFA), such as DHA, eicosapentaenoic acid (EPA), arachidonic acid (AA), and α-linolenic acid (ALA) in the form of differing lipid species (triglycerides, phospholipids, plasmalogens, cholesterol esters or gangliosides or cerebrosides) which can be specific to each pathology.

### 3. SUMMARY OF THE INVENTION

The present disclosure relates to disruption of an interaction between Aβ and neuronal lipids, such as DHA and EPA, where said disruption can be used to inhibit neurodegeneration associated with AD, PD, and synucleinopathies, such as dementia with Lewy bodies, DS and associated cognitive disorders, multiple system atrophy, and rare neuroaxonal dystrophies, such as NPC and Gaucher's disease. The disclosure further relates to assays for identifying agents that reduce interaction between amyloid peptide and neuronal lipids and accordingly can be useful as therapies for AD, PD and synucleinopathies, such as dementia with Lewy bodies, DS and associated cognitive disorders, multiple system atrophy, rare neuroaxonal dystrophies, such as NPC and Gaucher's disease. The disclosure further relates to the contribution of apolipoprotein E (ApoE) genotype to altered metabolism, maintenance and distribution of dietary lipids as cholesterol esters.

The disclosure further relates to methods and compositions for intranasal administration of fatty acids or lipids containing fatty acid acyl chains of dietary lipids, such as DHA and EPA, as bioactive agents for promoting central nervous system health and/or prevention or treatment of neurodegenerative disorders such as AD, PD, and synucleinopathies, such as dementia with Lewy bodies, DS and associated cognitive disorders, multiple system atrophy, and rare neuroaxonal dystrophies, such as NPC and Gaucher's disease. Specifically, therapeutic amounts of fatty acids, for example dietary polyunsaturated fatty acids such as DHA, EPA, or combinations thereof, are administered to a subject intranasally to promote central nervous system health, inhibit neurodegeneration, prevent or treat neurodegenerative disorders such as AD, PD, and synucleinopathies such as dementia with Lewy bodies, DS and associated cognitive disorders, multiple system atrophy, and rare neuroaxonal dystrophies, such as NPC and Gaucher's disease, and/or prevent, inhibit progression of, and/or treat cognitive impairment.

The disclosure further relates to the contribution of ApoE genotype to altered metabolism, maintenance and distribution of dietary lipids as cholesterol esters.

In certain non-limiting embodiments, a method of treatment is provided, wherein the interaction between Aβ and critical neuronal lipids, for example DHA, is blocked or inhibited in a subject in need of such treatment, for example but not limited to a subject who is elderly and/or suffers from mild cognitive impairment and/or suffers from Alzheimer's Disease.

In certain non-limiting embodiments, a method of blocking or inhibiting the interaction between DHA-CE and Aβ is provided, in a subject in need of such treatment. This interaction could be blocked with, for example but not limited to, small molecules, immunotherapeutics, soluble Aβ:DHA-CE complex mimetics, peptidomimetics, or nanoparticles. Interruption of the binding of DHA-CE (or other lipids) to Aβ is unlikely to effect the major functions of either lipids or Aβ which can allow avoidance of target and non-target based side effects. Immunotherapeutics (e.g., antibodies, including conventional light chain/heavy chain complexes as well as single chain antibodies and antibody fragments) could potentially be developed which target the Aβ:DHA-CE complex and thereby provide specificity for a pathogenic complex yet sparing normal function of Aβ and DHA-CE individually.

In certain non-limiting embodiments, an assay for identification of effective blockers of the DHA-CE(lipid)/Aβ interaction is provided. Small molecules, immunotherapeutics or nanoparticles could be screened for ability to block Aβ binding to DHA-CE.

In certain non-limiting embodiments, Aβ protein in form of soluble monomer, oligomer or fibril preparation can be bound to reacti-bind plates and exposed to detectably labeled lipid. After washing away non-bound lipid, the bound lipid (bound to Aβ) would be proportional to the detectable signal, for example a fluorescent signal which could be read with a fluorometer. Disruption of the Aβ: lipid interaction by small molecules, immune-therapeutics or nanoparticles would result in a decrease in the detectable (e.g., fluorescent) signal depending on efficacy and affinity rendering this assay amenable to high-throughput screening, as well as valuable for secondary assays to determine dose:response relationships. Lipid specificity for Aβ binding could also be determined using this assay as could the specific conformer/species of Aβ (i.e., fibril, oligomer, protofibril or monomer).

In certain non-limiting embodiments, lipid (e.g., DHA), in the form of phosphatidylethanolamine, which has a primary amine structural moiety in the lipid head group, can be bound to plates and exposed to detectably labeled AP. After washing away non-bound Aβ, the bound Aβ would be proportional to the detectable signal, for example a fluorescent signal which could be read with a fluorometer. Disruption of the Aβ: lipid interaction by small molecules, immune-therapeutics or nanoparticles would result in a decrease in the detectable (e.g. fluorescent) signal depending on efficacy and affinity rendering this assay amenable to high-throughput screening as well as valuable for secondary assays to determine dose:response relationships. Specificity of lipid for Aβ binding could also be determined using this assay as could the specific conformer/species of Aβ (i.e., fibril, oligomer, protofibril or monomer).

In certain non-limiting embodiments, ApoE, which contains primary amines in the amino acids of its protein sequence, can be bound to plates and exposed to detectably labeled Aβ in the presence or absence of lipid (e.g., DHA). After washing away non-bound Aβ, the bound Aβ would be proportional to the detectable signal, for example a fluorescent signal which could be read with a fluorometer. Disruption of the Aβ:lipid interaction by small molecules, immune-therapeutics or nanoparticles would result in a decrease in the detectable (e.g. fluorescent) signal depending on efficacy and affinity rendering this assay amenable to high-throughput screening as well as valuable for secondary assays to determine dose:response relationships. Specificity of lipid for Aβ binding could also be determined using this assay as could the specific conformer/species of Aβ (i.e., fibril, oligomer, protofibril or monomer).

A subject (or patient) can be human or non-human, such as but not limited to a non-human primate, rodent, dog, cat, horse, pig, rabbit, etc. In certain non-limiting embodiments the subject is a human subject suffering from one or more of AD, PD, a synucleinopathy (such as dementia with Lewy bodies), DS, multiple system atrophy, or a neuroaxonal dystrophies (e.g. NPC or Gaucher's disease). In certain non-limiting embodiments, a subject has dementia or mild cognitive impairment. In certain non-limiting embodiments, a subject exhibits high Aβ load by PET imaging.

In certain non-limiting embodiments, DHA supplementation is combined with anti-Aβ immunotherapy. Aβ immunotherapy has been largely unsuccessful due to the fact that at time of therapy, though Aβ is largely cleared from brain with immunotherapy, cognitive improvement has been modest at best. Based on the discovery disclosed herein, the critical amount of DHA or other lipid has already been leached from brain tissue and is not replenished from dietary sources. Therefore, it can be beneficial to increase dietary DHA or other lipid supplementation during Aβ immunotherapies or to counteract the synaptotoxic effects of excess Aβ.

Since dietary supplements of DHA and other lipids can have limited access to the brain due to the blood brain barrier, modes of supplementation that improve central nervous system access can be utilized, such as, but not limited to, lipid-based nanoparticles, lipoproteins, lipid emulsions, multifunctional liposomes or gene therapy-based alteration of lipid metabolism and distribution (e.g., provision of ApoE or DHA modifying enzymes including lipid transfer proteins, cholesterol ester transfer protein (CETP), lecithin-cholesterol acyltransferase (LCAT), or other components of reverse cholesterol transport and brain cholesterol metabolism.

In certain non-limiting embodiments, an intranasal pharmaceutical composition for treating a subject in need thereof is provided, comprising a therapeutically effective amount of lipid, including one or more polyunsaturated fatty acid, such as DHA, EPA, or combinations thereof. In certain non-limiting embodiments, said composition is administered to a subject intranasally to promote central nervous system health, inhibit neurodegeneration, prevent or treat neurodegenerative disorders and/or prevent, inhibit progression of, and/or treat cognitive impairment associated with AD, DS, PD, or synucleinopathies such as dementia with Lewy bodies and multiple system atrophy.

In certain non-limiting embodiments, said intranasal pharmaceutical compositions can include one or more triglyceride, phospholipid, plasmalogen, cholesterol ester, ganglioside, cerebroside, lipid-based nanoparticle, lipoprotein, lipid emulsion, multifunctional liposome or gene therapy-based alteration of lipid metabolism and distribution (e.g., provision of ApoE or DHA modifying enzymes including lipid transfer proteins, CETP, LCAT, or other components of reverse cholesterol transport or brain cholesterol metabolism.

In certain non-limiting embodiments, a method to promote central nervous system health, inhibit neurodegeneration, prevent or treat neurodegenerative disorders is provided, comprising administering a therapeutically effective amount of lipid intranasally. In certain non-limiting embodiments, said neurodegenerative disorder is mild cognitive disorder, Alzheimer's disease, or Down syndrome and associated cognitive disorders, Parkinson's disease or a synucleinopathy, including dementia with Lewy bodies, and multiple system atrophy.

### 4. BRIEF DESCRIPTION OF THE FIGURES

FIGS. 1A-1C. (A) Results are displayed as raw binding of relative fluorescent units (RFU). (B) Specific binding resulting from subtraction of background binding to di18:0PE. (C) Unlabeled or scrambled Aβ42 was incubated with plates with increasing amount of lipid and then FAM fluorescence was detected.

FIGS. 2A-2B. (A) Results are displayed as specific binding of relative fluorescent units (RFU) of Aβ42-Hilyute coated wells after subtraction of background binding (no ApoE, 0) in presence of increasing concentration of DHA (pmol/well). (B) Specific binding resulting from subtraction of background binding (no ApoE, 0) to ApoE coated wells in presence of either 22:6 containing lipids or control lipid 18:0.

FIG. 3. Administration (Tx) and testing schedule.

FIGS. 4A-4D. 10 days treatment with low dose SDPC. (A) After 10 days treatment (Tx) with low dose phosphatidylcholine (PC) containing docosahexaenoyl (22:6) and stearoyl (18:0) acyl chains, 18:0-22:6 PC; 1-stearoyl-2-docosahexaenoyl-sn-glycero-3-phosphocholine (CAS Number 59403-52-0; Synonyms: 1-octadecanoyl-2-(4Z,7Z,10Z,13Z,16Z,19Z-docosahexaenoyl)-sn-glycero-3-phosphocholine and PC(18:0/22:6(4Z,7Z,10Z,13Z,16Z,19Z))) (SDPC) intranasally, nesting score was assessed as described before (114). The number of nestlettes (from 3 initially placed in cage) was estimated and exact weight of remaining nestlettes was determined in grams (g) from initial 3 nestlettes (approximately 2 g). (B) Activity during novel object recognition (NOR) training was assessed after 10 days intranasal SDPC. Grooming behavior, free rearing, wall rearing and center crossing events are shown and were summed in total events. (C) Time spent with each identical object during NOR training. (D) Activity during Open Field testing 24 hours after NOR training (note, no difference in novel object discrimination (NOD) index was found. Standard error is shown for means of Saline treated APPsw+(control) (n=4); SDPC treated APPsw+(n=5) and wild type receiving no treatment (n=4). Significance was determined for p>0.05 using Student's t-test.

FIGS. 5A-5B. Nesting and activity after 30 days treatment with escalating dose SDPC. (A) After 30 days treatment (Tx) with low dose SDPC intranasally, nesting score was assessed as described (114). The number of nestlettes remaining (from 3 initially placed in cage) was estimated and exact weight of remaining nestlettes was determined in grams (g remaining) from initial 3 nestlettes (approximately 2 g). (B) Activity during NOR training was assessed after 10 days intranasal SDPC. Grooming behavior, free rearing, wall rearing and center crossing events were summed in total events. Standard error is shown for means of Saline treated APPsw+(control) (n=4); SDPC treated APPsw+(n=5) and wild type receiving no treatment (n=4). Significance was determined for p>0.05 using Student's t-test.

FIGS. 6A-6B. 30 days treatment with escalating dose SDPC. (A) Mice were placed in a box with two identical objects for 10 minutes on the training day 1. On training day 2, time spent with one displaced identical object was recorded to test for hippocampal function, but no discrimination was determined. One object was replaced with a novel object for testing (NOD index). The same data is shown to scale in FIG. 6B (Testing (immediate)). Twenty-four hours later (Testing (24 hours)), mice were placed in the same context with one of the original identical objects (Familiar) and a second novel object (Novel). The time spent with each object was recorded. (B) For testing, novel object discrimination (NOD index) is assessed using the equation [(time novel)/(time familiar+time novel)].

NOD index of 0.5 represents equal time with each object. NOD>0.5 represents more time spent with novel object. Standard error is shown for means of Saline treated APPsw+(control) (n=4); SDPC treated APPsw+(n=5) and wild type receiving no treatment (n=4). Significance was determined for p>0.05 using Student's t-test.

### 5. DETAILED DESCRIPTION OF THE INVENTION

For clarity and not by way of limitation, the detailed description of the invention is divided into the following subsections:

(1) Subjects for Treatment;

(2) Disruption of the interaction between Aβ and lipids;

(3) Screening assays for identifying blockers and/or inhibitors; and

(4) Lipid supplementation.

DHA, and other important membrane and signaling lipids, such as gangliosides (e.g. GM1), are highly hydrophobic by nature and bind to amyloid β peptide (Aβ) (7, 13, 14, 15, 16). Without being bound by theory, it is believed that (i) DHA is likely to bind in vivo to Aβ, associated with AD and DS and associated cognitive disorders, as well as to αS, the aggregated pathological hallmark of PD and other synucleinopathies, such as dementia with Lewy bodies, multiple system atrophy and rare neuroaxonal dystrophies, and (ii) this binding can prevent normal function of DHA in neurons.

### 5.1 Subjects for Treatment

A subject which can be treated can be a human or a non-human animal subject, such as, but not limited to, a dog, a cat, a horse, a mouse, a rat, a hamster, a guinea pig, a rabbit, a non-human primate, a goat, a sheep, or a cow.

In certain non-limiting embodiments, the subject is a human.

In certain non-limiting embodiments, the subject suffers from mild cognitive impairment.

In certain non-limiting embodiments, the subject suffers from Alzheimer's Disease.

In certain non-limiting embodiments, the subject suffers from Down Syndrome.

In certain non-limiting embodiments, the subject suffers from Parkinson's disease, or a synucleinopathy, such as dementia with Lewy bodies, multiple system atrophy or rare neuroaxonal dystrophies.

In certain non-limiting embodiments, a subject exhibits high Aβ load by PET imaging (Pittsburgh compound B; Flutemetamol/Vizamyl, florbetapir/Amyvid).

### 5.1.1 Alzheimer's Disease, Mild Cognitive Impairment

Subjects suffering from AD (or a non-human animal equivalent thereof) or mild cognitive impairment can benefit from blocking or inhibiting the interaction between amyloid β and lipids and/or intranasal lipid supplementation.

DHA cholesterol ester (DHA-CE) is specifically depleted in ventricular fluid in AD patients (25) suggesting that replacement of DHA-CE can prevent cognitive decline by preserving this lipid in neuronal membrane. In the Montine study, another unsaturated lipid 20:4 was generally spared in the ventricular fluid of AD patients indicating that the loss of poly-unsaturated lipids is not likely to be a general effect of oxidation of the double bonds. They also observed the up-regulation of 18:0 cholesterol ester. This can be a compensatory response for the loss of DHA-CE, since 18:0 is a simple lipid, which can be synthesized de novo, in contrast to DHA, which must be taken up through the diet or synthesized through an inefficient and metabolically expensive conversion from ALA.

The link between AD and atherosclerosis can be consistent with this hypothesis as well since it has been shown that dietary lipids required for neuronal function (i.e., DHA and EPA as cholesterol esters (DHA-CE and EPA-CE respectively), are sequestered by atherosclerotic plaques (26) potentially reducing availability in brain. Replacement of DHA directly to the brain through intranasal administration is a promising therapeutic strategy for delivery directly to the site of action in the central nervous system bypassing the BBB as well as absorption in peripheral tissues or circulating lipoproteins. In certain non-limiting embodiments, a polyunsaturated fatty acid having a particular acyl chain length can be administered according to the amyloid peptide fragment to be bound; for example, Aβ42, the longest and most amyloidogenic species of Aβ can favor binding to DHA (22:6); while Aβ40 can favor binding to intermediate length EPA (20:5) or AA (20:4), and Aβ38 can favor binding to linoleic acid (LA) (18:2) containing cholesterol esters. Accordingly, in certain non-limiting embodiments, a therapeutic amount of DHA can be administered intranasally for the reduction, prevention, and/or treatment of AD or mild cognitive impairment.

Apolipoprotein A4 is the strongest genetic risk factor for late onset AD. The protein encoded by the Apo ε (eplison) 4 genotype, ApoE4, predisposes one to development of AD (23, 24). It is the strongest risk factor for AD incidence and has been shown to alter responsiveness to certain therapeutics in clinical trials (23). ApoE binds to amyloid-β peptide (Aβ), the pathological hallmark in AD, with varying affinity depending on genotype (23) and coordinates lipid and cholesterol transfer from membranes to maturing lipoproteins interacting with lipid trafficking in neurons, between neurons and astrocytes or glia. ApoE4 can alter lipid metabolism and prevent delivery or alter metabolism or clearance of DHA or dietary lipids as cholesterol esters (DHA-CE and EPA-CE) to maintain or replenish critical lipids important for neuronal function and cognition, such as DHA, in brain tissue and cells. Therefore, having ApoE4 with altered lipid and Aβ binding capacities can predispose one to development of AD.

In summary, the interaction of three variables can lead to AD: 1) amount of (reserve) DHA or other critical neuronal lipids, 2) amount of Aβ which can serve as a lipid sink in molar amounts to lipid, especially dietary DHA and EPA, and 3) presence of the ApoE4 genotype, which can alter lipid metabolism and circulation of the cholesterol esters DHA-CE and EPA-CE, preventing maintenance or replenishment of neuronal lipids to functional cellular site. Cognitive decline can be expected after the loss of a critical mass of DHA or other important neuronal lipids, or sequestration of lipids in Aβ plaques or soluble oligomers, leading to the disruption of maintenance or replenishment of critical lipids. This can also be due to ApoE4 genotype and loss of function.

### 5.1.2 Down Syndrome

Those with Down Syndrome are very high risk for development of AD after age 50 years and represent a targeted population who may immediately benefit from intranasal docosahexaenoic acid. The DS population is unique in that they are high risk for AD and conversion can be studied longitudinally in a relatively short time. It has been shown that reduced levels of Aβ42 in plasma correlate with development of AD in an adult DS population (DSAD) perhaps due to accumulation of Aβ in the brain (96). This population (DS >50 years) is also a highly valuable study group since a relatively short term longitudinal study, 5 years, can capture effects of intranasal DHA on delaying onset of AD.

Further, DHA supplementation during gestation, and/or childhood development, and/or chronic/long term DHA/EPA treatment, can ameliorate DS symptoms and pathology. Both AD and DS share the defining pathological hallmark of AD, the accumulation of the synapto- and neuronal-toxic Aβ shown to effect neuronal function and eventually lead to neurodegeneration. Elevated levels of Aβ in DS occur as early as 22 weeks in utero primarily due to the triplication of APP on chromosome 21 in DS (80). AD-like pathology has been observed as early as age 12 and is nearly ubiquitous in 40 year old adults with DS and AD dementia (DSAD) manifests only after the 5th decade of life in the majority of DS adults. DS is defined by trisomy of chromosome 21 encoding 161 genes, several of which have been shown to be overexpressed as proteins in DS and DSAD compared to age matched controls.

### 5.1.3 Parkinson's Disease, Synucleinopathies

Subjects suffering from PD and other synucleinopathies such as dementia with Lewy bodies, multiple system atrophy and rare neuroaxonal dystrophies, such as NPC and Gaucher's disease (89) can benefit from blocking or inhibiting the interaction between αS and lipids and/or intranasal lipid supplementation. PD pathology is characterized by accumulation of αS aggregates and degeneration of the dopaminergic neurons of the substantia nigra. αS is a 14 KDa, hydrophobic protein with alpha helical structure which aggregates into larger oligomeric species such as tetramer in vitro (67, 71, 76, 109). The alpha helical nature of αS is enhanced by lipid binding (74, 104). The dysregulation of glucocerebroside lipids (sphingoid-base lipids containing a glucose head group) by mutation in the degrading enzyme β-glucocerebrosidase (GBA). The resulting accumulation of glucocerebroside in the lysosome causes lysosomal storage disorder proposed to lead to neurodegeneration in Gaucher's disease. Loss of function mutations in GBA have also been associated with PD.

Glucocerebrosides contain sphingosine, glucose and a fatty acid of varying length. Polyunsaturated fatty acids (n−3) such as, but not limited to, DHA (22:6) and EPA (20:5) are dietary lipids which cannot be synthesized by mammals except through an inefficient and metabolically expensive conversion from ALA. Therefore, the dietary absorption of DHA and EPA are critical for maintaining sufficient levels of these lipids. Further, these critical lipids are likely to be tightly regulated and perhaps scavenged for re-use in intracellular membranes. The sphingosine lipid backbone contains an amino alcohol and αS has been shown to complex with polyamines suggesting affinity for primary amines (76). αS also binds cholesterol and redistributes cholesterol disrupting the liquid-ordered phase of the membrane and perhaps lowering the energetics of inserting and removing lipids from a bi-layer (105). Similar altered membrane fluidity has been suggested for amyloid β-peptide in Alzheimer's disease (84, 106).

Without being bound by theory, αS may act as a lipid scavenger which would bind glucocerebrosides, especially species with polyunsaturated fatty acid acyl chains such as DHA and EPA. Binding may result in complex formation with Apolipoprotein E which has been shown to be genetically linked to dementia in pure synucleinopathies (103). ApoA-I has been proposed to associated with membranes allowing free movement of two amphipathic α-helices in a hinge like manner (97). This “hinge” domain may be able to remove and insert lipids bound to αS into the outer leaflet of the bi-layer of cellular membranes such synaptic vesicle membranes or membranes of the lysosome. It has been shown that αS monomer has been shown to increase membrane area after insertion of alpha helix consistent with this hypothesis (99). It is possible that ApoE and αS may work in concert to regulate synaptic membrane composition and vesicle size, which is tightly regulated. Synaptic vesicle release and endocytosis has been shown to be altered by overexpression of αS (92). There are three synuclein isoforms which differ by size, αS:140 amino acids, 126 amino acids, and 112 amino acids, but share high homology in the N-terminal region which binds acidic lipids (102). Differing lengths could reflect different lengths of lipids such as αS 140 binding the longest glucocerebroside containing DHA (22:6); 126 binding intermediate length EPA (20:5) or AA (20:4) and 112 binding to LA (18:2) containing glucocerebrosides. This mechanism is similar to the proposed binding of Aβ to cholesterol esters containing DHA, EPA and AA.

Further, with ageing and accumulation of free radicals, polyunsaturated fatty acids like DHA and EPA may be lost and Aβ or αS may be in molar excess of lipids which bind and promote helix formation. In absence of these lipids, unbound Aβ and αS may, in a disordered state, form oligomers and higher order aggregates. As Aβ aggregates and looses functional lipid trafficking and scavenging activities, it also gains a toxic function in the cells. As αS aggregates and loses function, lipids accumulate in the lysosome leading to toxic gain of function leading to neurodegeneration as in Gaucher's disease. By replacing molar amounts of lipid, Aβ and αS may be kept in equimolar amount with DHA or EPA containing cholesterol esters and glucocerebrosides respectively preventing the unbound disordered state from forming and leading to aggregation.

### 5.1.4 Niemann-Pick Type C Disease

In certain non-limiting embodiments, Niemann-Pick patients with mutations in NPC1/2 can benefit from blocking or inhibiting the interaction between NPC1 and/or NPC2 and lipids and/or intranasal lipid supplementation. The distribution of lipids is the key feature of Niemann-Pick disease which is hallmarked by accumulation of cholesterol, but has also been associated with Aβ deposition and ApoE mutations (88).

Though little is known about the function of causative mutations in NPC1 and NPC2, these proteins both show cholesterol binding sites and similarity to apolipoproteins (69, 94). These proteins NPC1/2 may act in concert with lipid recognition proteins Aβ and αS to control lipid distribution in the neuron.

### 5.2 Disruption of the Interaction Between Aβ and Lipids

In certain non-limiting embodiments, a method of treatment is provided, wherein the interaction between Aβ or αS and lipids is blocked or inhibited in a subject in need of such treatment.

In certain non-limiting embodiments, a method of treatment is provided, wherein the interaction between Aβ or αS and neuronal lipids, for example DHA and/or EPA, is blocked or inhibited in a subject in need of such treatment.

In certain non-limiting embodiments, a method of blocking or inhibiting the interaction between DHA-CE and Aβ is provided, in a subject in need of such treatment.

In certain non-limiting embodiments, the interaction between Aβ or αS and neuronal lipids and/or the interaction between DHA-CE and Aβ is blocked or inhibited by a blocker or an inhibitor.

In certain non-limiting embodiments, the blocker or the inhibitor includes at least one of a small molecule, an immunotherapeutic, a soluble Aβ:DHA-CE complex mimetic, a peptidomimetic, and/or a nanoparticle.

In certain non-limiting embodiments, immunotherapeutics include at least one of antibodies, conventional light chain/heavy chain complexes, single chain antibodies and antibody fragments. Immunotherapeutics (e.g., antibodies, including conventional light chain/heavy chain complexes as well as single chain antibodies and antibody fragments) can be developed, which target the Aβ:DHA-CE complex and thereby provide specificity for a pathogenic complex yet sparing normal function of Aβ and DHA-CE individually.

Interruption of the binding of DHA-CE (or other lipids) to Aβ is unlikely to effect the major functions of either lipids or Aβ which can allow avoidance of target and non-target based side effects.

In certain non-limiting embodiments, the inhibitor interferes with binding between a lipid, for example but not limited to DHA or EPA, and Aβ at SEQ ID NO:1.

In particular non-limiting embodiments, the inhibitor binds to Aβ at SEQ ID NO:1. In particular non-limiting embodiments, the inhibitor binds to SEQ ID NO:1. In particular non-limiting embodiments, the inhibitor competitively binds with an antibody specific for SEQ ID NO:1 for binding to Aβ.

In certain non-limiting embodiments, the inhibitor interferes with binding between a lipid, for example but not limited to DHA or EPA, and Aβ at subregion FFAEDVGSNKGAIIGLMVGGVV (SEQ ID NO:5). In particular non-limiting embodiments, the inhibitor binds to Aβ at FFAEDVGSNKGAIIGLMVGGVV (SEQ ID NO:5). In particular non-limiting embodiments, the inhibitor binds to FFAEDVGSNKGAIIGLMVGGVV (SEQ ID NO:5). In particular non-limiting embodiments, the inhibitor competitively binds with an antibody specific for FFAEDVGSNKGAIIGLMVGGVV (SEQ ID NO:5) for binding to Aβ.

In certain non-limiting embodiments, the inhibitor interferes with binding between a lipid, for example but not limited to DHA or EPA, and αS at SEQ ID NO:2. In particular non-limiting embodiments, the inhibitor binds to αS at SEQ ID NO:2. In particular non-limiting embodiments, the inhibitor binds to SEQ ID NO:2. In particular non-limiting embodiments, the inhibitor competitively binds with an antibody specific for SEQ ID NO:2 for binding to αS.

In certain non-limiting embodiments, the inhibitor interferes with binding between a lipid, for example but not limited to DHA or EPA, and αS at subregion GAVVTGVT (SEQ ID NO:6). In particular non-limiting embodiments, the inhibitor binds to αS at GAVVTGVT (SEQ ID NO:6). In particular non-limiting embodiments, the inhibitor binds to GAVVTGVT (SEQ ID NO:6). In particular non-limiting embodiments, the inhibitor competitively binds with an antibody specific for GAVVTGVT (SEQ ID NO:6) for binding to αS.

### 5.3 Screening Assays for Identifying Blockers and/or Inhibitors

In certain non-limiting embodiments, an assay for identifying effective blockers of the DHA-CE(lipid)/Aβ interaction is provided. In certain non-limiting embodiments, an assay for screening small molecules, immunotherapeutics, and/or nanoparticles for their ability to block Aβ binding to DHA-CE is provided.

In certain non-limiting embodiments, Aβ protein in form of soluble monomer, oligomer, fibril preparation (27), and/or a peptide fragment of Aβ which is not the complete Aβ protein, e.g. a peptide comprising either SEQ ID NO:1 or a subsequence thereof, for example, an up to 50-mer or up to 30-mer peptide comprising FFAEDVGSNKGAIIGLMVGGVV (SEQ ID NO:5) is bound to reacti-bind plates and exposed to detectably labeled lipid (for example, fluorescent (e.g., BODIPY)-tagged lipid (i.e., DHA, 22:6). After washing away non-bound lipid, the bound lipid (bound to Aβ) is proportional to the detectable signal. In certain non-limiting embodiments, the detectable signal is a fluorescent signal, which could be read with a fluorometer. In certain non-limiting embodiments, disruption of the Aβ: lipid interaction by small molecules, immunotherapeutics, soluble Aβ:DHA-CE complex mimetics, peptidomimetics, and/or nanoparticles results in a decrease in the detectable signal.

In certain non-limiting embodiments, disruption of the Aβ: lipid interaction by small molecules, immune-therapeutics or nanoparticles results in a decrease in the detectable signal depending on efficacy and affinity rendering this assay amenable to high-throughput screening. In certain non-limiting embodiments, said assay is used to determine dose:response relationships. In certain non-limiting embodiment, said assay is used to determine the specificity of lipid for Aβ binding or the specific conformer/species of Aβ (i.e., fibril, oligomer, protofibril, or monomer).

In certain non-limiting embodiments, lipid (e.g., DHA), in the form of phosphatidylethanolamine (“PE”), which has a primary amine structural moiety in the lipid head group, is bound to plates and exposed to detectably labeled Aβ (for example, but not limited to, fluorescently labeled Aβ, e.g., Aβ labeled with FAM, HiLyte Fluor™ or TAMRA, Anaspec Freemont, Calif.). After washing away non-bound Aβ, the bound Aβ is proportional to the detectable signal. In certain non-limiting embodiments, the detectable signal is a fluorescent signal, which could be read with a fluorometer. In certain non-limiting embodiments, disruption of the Aβ: lipid interaction by small molecules, immunotherapeutics, soluble Aβ:DHA-CE complex mimetics, peptidomimetics, and/or nanoparticles results in a decrease in the detectable signal.

In certain non-limiting embodiments, disruption of the Aβ: lipid interaction small molecules, immunotherapeutics, soluble Aβ:DHA-CE complex mimetics, peptidomimetics, and/or nanoparticles results in a decrease in the detectable signal depending on efficacy and affinity rendering this assay amenable to high-throughput screening. In certain non-limiting embodiments, said assay is used to determine dose:response relationships. In certain non-limiting embodiment, said assay is used to determine the specificity of lipid for Aβ binding or the specific conformer/species of Aβ (i.e., fibril, oligomer, protofibril, or monomer).

In certain non-limiting embodiments, ApoE, which contains primary amines in the amino acids of its protein sequence, is bound to plates and exposed to detectably labeled Aβ. (for example, but not limited to, fluorescently labeled Aβ, e.g., Aβ labeled with FAM, HiLyte Fluor™ or TAMRA, Anaspec Freemont, Calif. in the presence or absence of lipid (e.g., DHA). After washing away non-bound Aβ, the bound Aβ is proportional to the detectable signal.

In certain non-limiting embodiments, disruption of the Aβ: lipid interaction by small molecules, immunotherapeutics, soluble Aβ:DHA-CE complex mimetics, peptidomimetics, and/or nanoparticles results in a decrease in the detectable signal depending on efficacy and affinity rendering this assay amenable to high-throughput screening.

In certain non-limiting embodiments, said assay is used to determine dose:response relationships. In certain non-limiting embodiment, said assay is used to determine the specificity of lipid for Aβ binding or the specific conformer/species of Aβ (i.e., fibril, oligomer, protofibril, or monomer).

In certain non-limiting embodiments the invention provides for an analogous assay for inhibitors of the interaction between lipids and αS, where αS, or an αS peptide, e.g. a peptide comprising SEQ ID NO:2, or comprising SEQ ID NO:3, or comprising SEQ ID NO:4, or comprising GAVVTGVT (SEQ ID NO:6), or an up to 30-mer or up to 50-mer peptide comprising said sequences, may be used instead of the Aβ protein or peptide fragments bound to the plate.

### 5.4 Lipid Supplementation

In certain non-limiting embodiments of the invention, the pathological interaction between dietary lipids (DHA and EPA) and Aβ can be inhibited by administration of an exogenous formulation of DHA or EPA containing lipid, which can bind competitively to endogenous Aβ. This can result in either 1) unbinding of essential DHA or EPA freeing lipids from Aβ for endogenous function, or 2) replacement of Aβ depleted endogenous lipid function by exogenously administered DHA and EPA containing lipids, restoring the “critical mass” of bioavailable DHA or EPA lipids required for brain function. Either 1) or 2) can result in rescued neuronal and brain function known to be aberrant in AD. This (1 or 2 above) can be accomplished by aggressive supplementation with, for example but not limited to, DHA or EPA as the fatty acid (acyl-chain) component of the phospholipids, phosphatidylcholine (PC), phoshatidylethanolamine (PE), free fatty acids (ethyl esters), triglycerides, phosphatidylserine (PS), phosphatidylserine, cholesterol-esters (CE), and/or plasmalogens.

In certain non-limiting embodiments, DHA supplementation is combined with anti-Aβ immunotherapy. Based on the discovery disclosed herein, the critical amount of DHA or other lipid has already been leached from brain tissue and is not replenished from dietary sources. Therefore, it can be beneficial to increase DHA or other lipid supplementation during Aβ immunotherapies (i.e., Solanezumab, BIIB037/Aducanumab, Crenezumab, Bapineuzumab, Gantenerumab) or to counteract the synaptotoxic effects of excess Aβ.

Since the administration of DHA through 18:0-22:6 PC can directly be incorporated into ApoE/cholesterol metabolism in the brain, it can be effective for delivery of exogenous DHA, EPA, or dietary lipids into the correct brain metabolic pathways relevant in AD. The use of phosphatidylcholine containing DHA, (1-stearoyl-2-docosahexaenoyl-sn-glycero-3-phosphocholine (18:0-22:6 PC; SDPC) can be an effective way to rescue neuronal and brain function, because it can directly target the cholesterol homeostasis in the brain, through maturation of ApoE-containing high density lipoprotein particles (ApoE-HDL). Brain cholesterol metabolism is largely isolated from peripheral cholesterol metabolism, due to inability of cholesterol to pass the blood brain barrier (129). ApoE-HDL is secreted by astrocytes in the brain as is lecithin:cholesterol acyltransferase (LCAT). Lecithin is the original name for phosphatidylcholine, which is the major substrate for LCAT, the enzyme responsible for transferring an acyl chain (such as DHA or EPA) from PC to cholesterol. LCAT uses ApoE-HDL as a substrate and ApoE is a major activator of LCAT in the CNS. Therefore, LCAT can play a major role in the maturation of ApoE-HDL (129). Genetic variants of ApoE are the greatest risk factor for sporadic AD. Moreover, LCAT is increased in AD. These findings suggest pathological dysregulation of this pathway (130). Therefore, PC containing DHA as an acyl chain (18:0-22:6 PC, above) can feed directly into this pathway leading to ApoE-HDL maturation through incorporation of DHA from exogenously administered DHA containing lipid. Other cholesterol metabolizing/transfer proteins which can be involved are cholesterylester transfer protein (CETP) and phospholipid transfer protein (PLTP) (130, 131).

Since dietary supplements of DHA and other lipids can have limited access to the brain due to the blood brain barrier, modes of supplementation that improve central nervous system access can be utilized. In certain non-limiting embodiments, modes of supplementation include at least one of lipid-based nanoparticles, lipoproteins, lipid emulsions, multifunctional liposomes, and gene therapy-based alteration of lipid metabolism and distribution. In certain non-limiting embodiments, gene therapy-based alteration of lipid metabolism and distribution includes alteration of ApoE or DHA modifying enzymes, including lipid transfer proteins, CETP, LCAT, or other components of reverse cholesterol transport or brain cholesterol metabolism.

Also disclosed herein are methods and compositions that avoid the aforementioned problems, wherein therapeutic amounts of fatty acids or lipids containing fatty acid acyl chains of dietary lipids, for example dietary polyunsaturated fatty acids (PUFA) such as DHA, EPA, or combinations thereof, are administered to a subject intranasally to promote central nervous system health, inhibit neurodegeneration, prevent or treat neurodegenerative disorders such as AD, PD, synucleinopathies such as dementia with Lewy bodies, multiple system atrophy, neuroaxonal dystrophies, or neurodegeneration associated with DS, and/or prevent, inhibit progression of, and/or treat cognitive impairment.

### 5.4.1. Dose

In certain non-limiting embodiments, daily dose can be based on the American Heart Association guidelines for consumption of fish or fish oil supplementation orally in humans ranging from 250 mg-4000 mg per day (prescription Lovaza®) for patients with high triglyceride level (16).

In certain other embodiments, lower doses, for example, human doses that are less than 250 mg per day, or less than 200 mg per day, or less than 100 mg per day, or between about 100-200 mg per day, or between about 100-150 mg/day, can be used.

In certain non-limiting embodiments, a daily dose of between about 20 and 55 mg per pound body weight, or between about 5 and 15 mg per pound of body weight, can be administered to a dog or cat.

In certain other embodiments, a murine daily dose can be 0.72 g-11.52 g fish oil containing 32.7% EPA:32.7% DHA/kg diet chow (16). Doses for other species can be calculated using interspecies conversion calculations known in the art.

### 5.4.1 Intranasal Delivery

Dysfunction of the lipid redistribution scheme described above can lead to neurodegeneration associated with these diseases. The distribution of PUFA within the intracellular membrane and plasma membrane is critical for neuronal function of lipid rafts, membrane trafficking, signal transduction and conduction and myelination. Therefore, intranasal supplementation to replace critical lipids, can slow disease progression. In all mentioned pathologies, lipid supplementation and/or disruption of the binding between lipids and Aβ or αS can be used alternatively or in combination as a biotherapeutic.

In certain non-limiting embodiments, the present invention provides for an intranasal device comprising a therapeutic amount of a polyunsaturated fatty acid such as DHA, EPA, or a combination thereof, optionally together with a pharmaceutically acceptable excipient. An intranasal device, for example and not by limitation, may have a reservoir containing a polyunsaturated fatty acid such as DHA, EPA, or a combination thereof, a means for propelling the polyunsaturated fatty acid(s) out of the device and through the nostril, and a conduit having an aperture at its distal end to be placed in or near the nostril through which the polyunsaturated fatty acid(s) may be propelled upon activation of the device. In certain non-limiting embodiments the reservoir may be pressurized to a level higher than standard atmospheric pressure. In certain non-limiting embodiments, the device may be configured for human use or for use in a non-human animal such as a dog, a cat, or a horse. In certain non-limiting embodiments, the polyunsaturated fatty acid(s) is the only active ingredient contained in the device, any other components being inactive ingredients/excipients or preservatives. Any intranasal delivery device known in the art can be used to practice the disclosed methods (123). One non-limiting example of a suitable device is the Aptar Pharma nasal spray pump.

In certain non-limiting embodiments, the fatty acid(s) or lipids containing fatty acids, for example dietary polyunsaturated fatty acids such as DHA, EPA, or combinations thereof, are comprised in a pharmaceutical formulation suitable for intranasal delivery.

In certain non-limiting embodiments, said fatty acid(s) are provided in the form of lipid-based nanoparticles, lipoproteins, lipid emulsions, and/or multifunctional liposomes and/or can optionally be combined with means for gene therapy or protein-based alterations of lipid metabolism and distribution, such as, but not limited to, ApoE or DHA modifying enzymes including lipid transfer proteins, CETP, LCAT, or other components of reverse cholesterol transport or brain cholesterol metabolism.

Certain non-limiting embodiments provide for a formulation suitable for intranasal administration comprising an amount of dietary PUFA, such as DHA, EPA, or combinations thereof effective in promoting central nervous system health, inhibiting neurodegeneration, preventing or treating neurodegenerative disorders such as AD, PD, synucleinopathies such as dementia with Lewy bodies, multiple system atrophy, neuroaxonal dystrophies, or neurodegeneration associated with DS, and/or preventing, inhibiting progression of, and/or treating cognitive impairment. In certain non-limiting embodiments, the fatty acid(s) or lipids containing fatty acid acyl chains of dietary lipids is/are the sole therapeutic agent in the formulation; for example, the formulation lacks a second pharmaceutical active agent (e.g., neurotherapeutic agent). Other preparations can be from DHA enriched egg for phosphatidylcholine based preparations. Other lipid preparations can be synthesis of specific lipids containing DHA or EPA, which are determined to be efficacious.

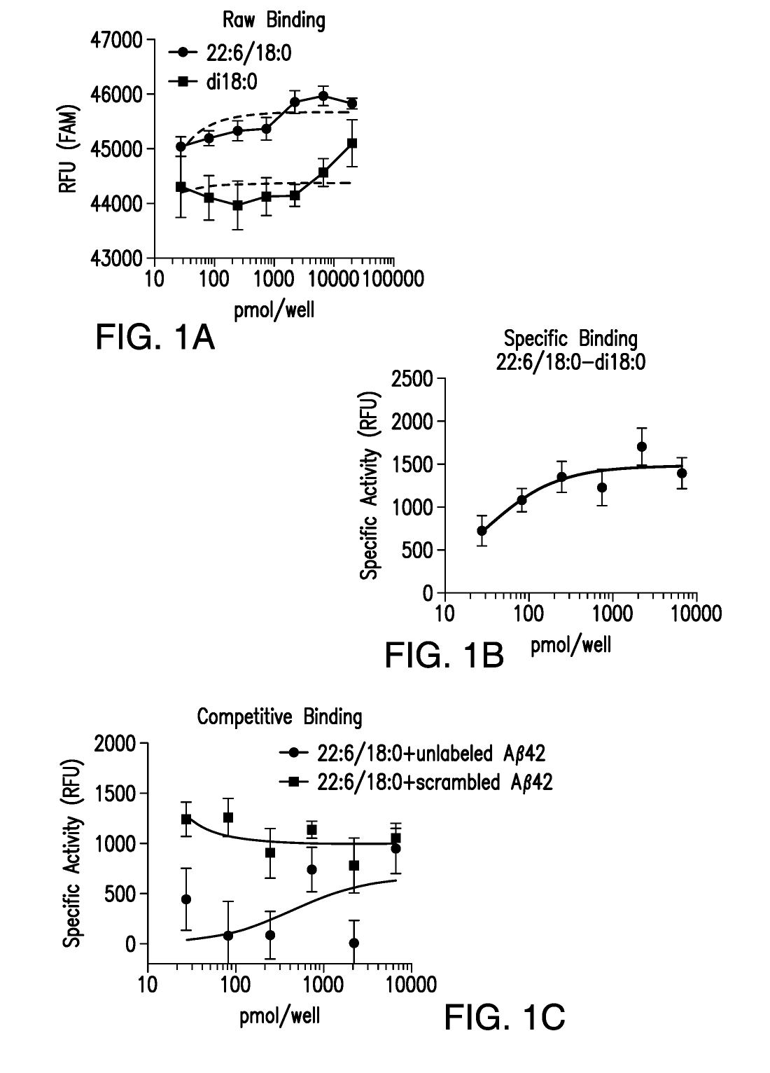

Risk are minimal for doses near currently common practice level of dietary supplementation. Preliminary experiments indicate that Aβ bound DHA with an estimated Kd of 300 nM (FIG. 1B), much lower than the estimated critical micelle concentration (CMC) for DHA (60 μM) (98). At lipid concentrations near the CMC in vitro, aggregation of Aβ was enhanced toward a non-fibril oligomer (85, 86). It is not clear if this oligomeric species is toxic, however, this may not be physiologically relevant, since lipids in a membrane are essentially at a concentration greater than the CMC. In certain non-limiting embodiments, for intranasal administration, the source of DHA and EPA is of high purity, for example, but not limited to, DHA and EPA prepared from algae to avoid fish oil contaminants, which can lead to allergic reaction. Other preparations can be from DHA enriched egg for phosphatidylcholine based preparations. Other lipid preparations can be synthesis of specific lipids containing DHA or EPO, which are determined to be efficacious.

Precise dosing can be controlled using specific intranasal spray devices, such as Unitdose or Biodose® liquid, which are available from Aptar Pharma. Due to the potential long (>2 year) half-life of DHA in brain (72, 127, 128), daily administration may not be required, but efficacy of weekly or monthly administration may be compared in clinical trials for both self administration and administration in the clinic when controlled for patient compliance. Preliminary studies indicate sub-micromolar affinity of Aβ for DHA (300 nM).

In certain non-limiting embodiments, intranasal administration of fatty acid, e.g., DHA, EPA, or lipids containing DHA and/or EPA, or a combination thereof, can be performed once daily, twice daily, three times daily, or four times daily, at least five times weekly, every other day, at least twice weekly, twice weekly, once a week, once a month, or twice a month. In certain non-limiting embodiments, the duration of treatment can be at least one month, at least three months, at least 6 months, six months, at least one year, one year.

Intranasal delivery of lipids may optionally be combined with other treatment modalities described herein, including but not limited to, non-lipid agents that inhibit or interfere with the Aβ-lipid interaction.

### 6. EXAMPLE 1: SCREENING ASSAYS FOR IDENTIFYING BLOCKERS AND/OR INHIBITORS

### 6.1 Materials and Methods

Lipid Binding Assay.

Maleic Anhydride Activated plates (Pierce Amine-binding, 96-well plates, Thermo Scientific) were washed in wash buffer (PBS: Phosphate buffered saline, 0.15 M sodium chloride, pH 7.2 containing 0.05% Tween-20 Detergent, PBST, Thermo Scientific) 4 times to activate reactive maleic anhydride functional group. Amine containing lipids were PE containing docosahexaenoyl (22:6) and stearoyl (18:0) acyl chains (22:6/18:0 PE) or two stearoyl acyl chains (di18:0 PE) were from Avanti Polar Lipids. 22:6/18:0 PE was obtained in chloroform, dried down and solubilized at 200 pmol/μL in 1% n-octylglucoside (NOG, Santa Cruz) in PBS and sonicated for 5 minutes. di18:0 PE was obtained as a powder, solubilized at 200 pmol/μl in 1% NOG and bath sonicated for 5 minutes. Lipids were incubated at a volume of 100 μL in activated maleic anhydride plates at increasing concentration at 4° C. in PBS/1% NOG. After incubation, lipids were removed and SuperBlock Blocking Buffer/PBS (Thermo) was added at a volume of 200 μL/well for 1 hour at room temperature. Plates were then washed 4 times with PBST (Thermo Scientific). Fluorescently labeled amyloid β-peptide (SensoLyte Fluorescent β-Amyloid1-42 Sampler Kit, Anaspec) was prepared with 5 μL Component B, Solvent for β-amyloid (Anaspec) as per the commercial protocol and then diluted in deionized water to a concentration of 100 μM. Aβ42-FAM was incubated at 200 nM in 100 ul SuperBlock/PBS overnight at 4° C. Plates were then washed 4 times in PBST and fluorescence was detected using Tecan Infinite 200 at wavelengths 494/521 (excitation, emission) using the Optimal Gain setting.

Competition.

Scrambled Aβ or unlabeled Aβ42 peptides (Anaspec) were diluted to 100 μM as above and then diluted in SuperBlock Blocking buffer to 1 μM, incubated for 1 hour at room temperature. Plates were washed and FAM fluorescence was read as above.

ApoE Binding Assay.

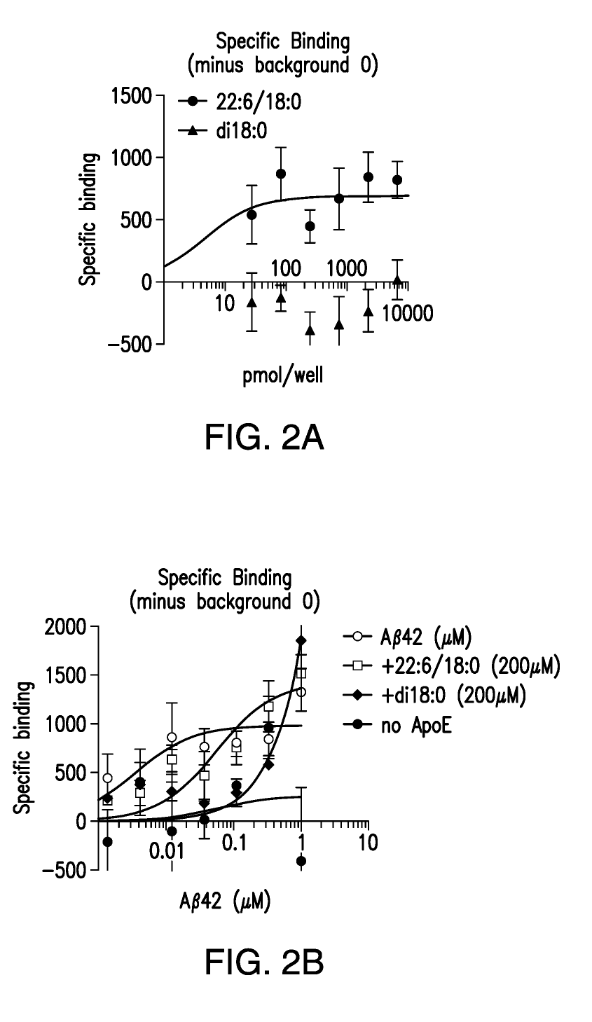

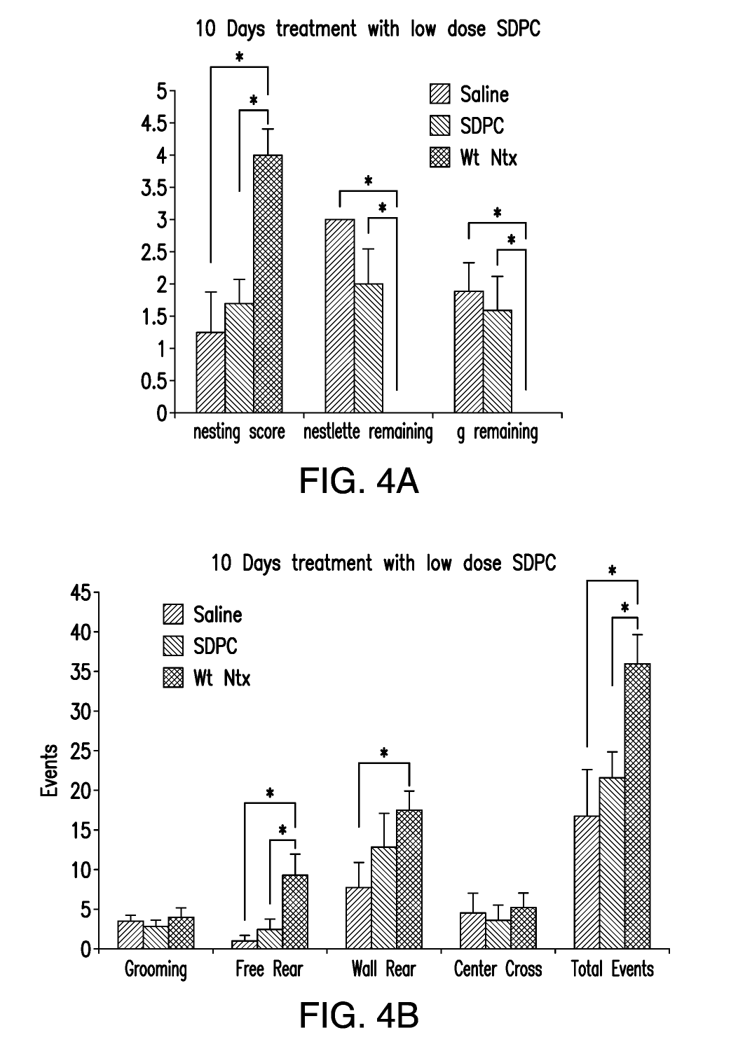

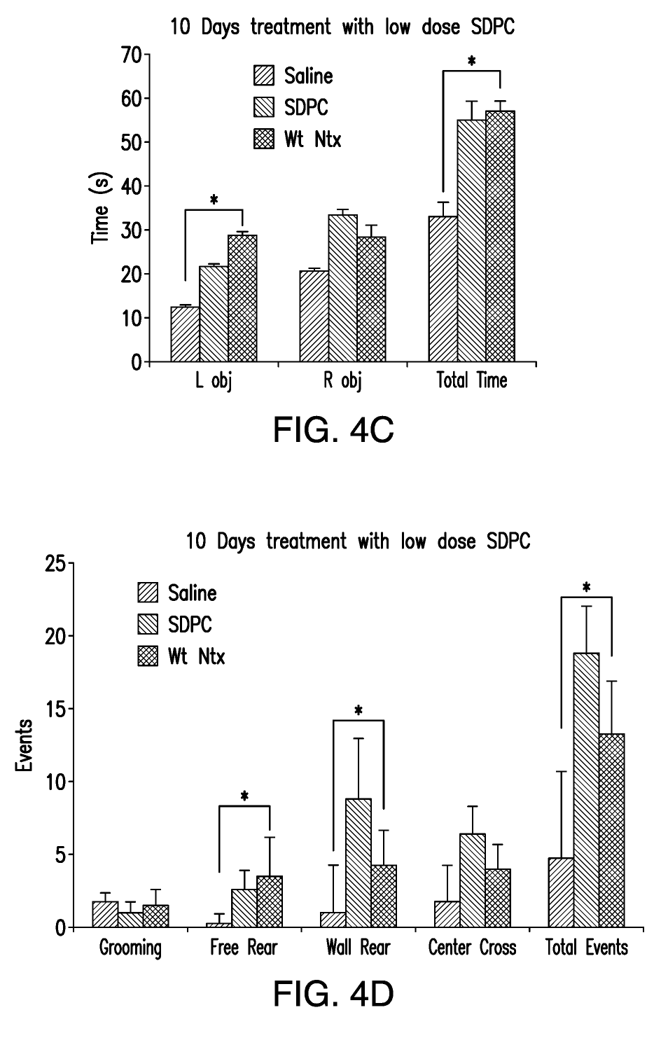

Plates were prepared as above and incubated with a constant amount of apolipoprotein E (ApoE, rPeptide) at 12.5 pmol/well FIG. 2A or 4 pmol/well FIG. 2B for 1 hour at room temperature with shaking. Plates were then blocked for 1 hour and washed with PB ST. Aβ labled with HiLyte (Anaspec) was prepared as above and mixed with increasing amount of lipid in constant concentration of NOG (0.0034%) in SuperBlock Blocking buffer and incubated overnight at 4° C. Binding was read as above at 503/528 excitation/emission).

Statistics.

Assays were done in triplicate wells and reported as mean+/−standard error. Binding kinetics and best fit curve fitting were accomplished using Prism Graphpad software.

### 6.2 Results

Aβ Binding to DHA.

Lipid containing long chain polyunsaturated fatty acid 22:6, docosahexaenoic acid (DHA) and an amine containing headgroup, phosphatidylethanoloamine (PE) was bound to maleic anhydride activated plates which bind to free primary amine functional groups at neutral and alkaline pH. All binding and washing steps were done in PBS, PBST and SuperBlock PBS to maintain pH at 7.2. A control acyl chain lipid hypothesized not to bind to Aβ peptide was 18:0, stearic acid containing PE (di18:0). After lipids were bound and unreacted binding sites were blocked using SuperBlock, Aβ42 peptide fluorescently tagged with FAM (Anaspec) was incubated with lipid bound wells. As expected, Aβ42 di 18:0 showed very low binding activity to Aβ42 at near background levels at concentrations 10,000 pmol/well and below (FIGS. 1A and 1B). Only at the highest concentrations did modest binding occur. However, Aβ42-FAM bound lipids containing 22:6 bound to the plate and displayed saturable one-site binding (FIGS. 1A and 1B). The dissociation constant Kd was calculated to be 300 nM. Specific binding was calculated by subtracting background binding of di18:0 PE. Binding could be competitively disrupted by unlabeled Aβ42 peptide (5×, 1 μM), but not robustly disrupted with comparable concentration of scrambled sequence Aβ42 peptide (FIG. 1C). This is a clear demonstration that the specific binding of dietary lipid DHA to Aβ is specific and robust.

Further studies may be executed to determine the requirement of double bonds and acyl chain length for Aβ binding. It is also possible that other commonly found Aβ species Aβ38, Aβ40, Aβ42, are specific for different acyl chain lengths with specific unsaturation requirements. Specifically, experiments will be performed to evaluate whether Aβ38 binds arachidonic acid containing lipids (20:4); Aβ40 binds eicosapentaenoic acid (20:5) containing lipids and Aβ42 binds selectively to DHA 22:6 containing lipids.

ApoE Binding.

ApoE coated plates (maleic anhydride activated plates bound to ApoE peptide which contains primary amine containing amino acids in the protein sequence) were incubated with fluorescent Aβ42-Hilyte in presence of increasing concentration of 22:6 or 18:0. Specific binding was determined by subtracting non-specific binding to the plate in absence of ApoE (no ApoE, 0). Aβ-Hilyte bound ApoE in presence of 22:6 containing lipid, but not when co-incubated with 18:0 containing lipids indicating the specificity for Aβ:ApoE:lipid binding complex (FIG. 2A). Interestingly, Aβ-FAM bound ApoE coated plates with a lower Kd (dissociation constant) in absence of DHA lipid (FIG. 2B) indicating DHA shifts the binding constant, reducing Aβ:ApoE binding. Aβ42-FAM bound apoE only very minimally in presence of 18:0 and only at very high concentrations of Aβ.

### 6.3 Discussion

DHA, and other important membrane and signaling lipids such as the ganglioside, GM1, are highly hydrophobic by nature and interact with Aβ42 (7,13-16). Pathological levels of Aβ in AD may then serve as a “lipid sink” which would leach critical lipids (potentially including but not limited to DHA) out of neuronal membranes causing both acute synaptotoxic and chronic neurotoxic phenomenon leading to cognitive decline. The effect of this lipid sink could explain the delay between Aβ accumulation in the brain in the soluble and deposited form, decades before clinical symptoms manifest. Depending on the abundance of DHA and other critical lipids in neurons, it would require varying amounts of Aβ as well as varying amounts of time to sequester, remove or “sink” a critical mass of DHA from brain tissue before neuronal function is compromised. Resistance to Aβ induced cognitive decline often referred to as “cognitive reserve” (17) could be explained by reserve levels of DHA or other lipids in the brain or intake of these dietary lipids. For example, a patient with higher levels of DHA, or higher dietary intake, would require higher levels of Aβ to accumulate and sequester enough DHA or other lipid before affecting neuronal function and subsequent synapse and neuron loss. The variability and poor temporal correlation between Aβ accumulation and cognitive dysfunction is also consistent with a long in vivo half-life of DHA in human brain, which is estimated to be greater than 2 years (72, 127, 128). This is consistent with the lowered risk of AD in populations which have a Mediterranean diet consisting of high intake of dietary lipids such as DHA (18,19). Similarly, this would explain why clearance of Aβ alone does not correlate with improved cognitive function (20).

Apolipoprotein E (APOE) ε4 allele is the strongest genetic risk factor for late onset AD (21,22). The protein encoded by the APOEε4 genotype, apoE4, predisposes one to development of AD (23, 24) It is the strongest risk factor for AD incidence and has been shown to alter responsiveness to certain therapeutics in clinical trials (23). ApoE4 increases Aβ deposition relative to other isoforms of apoE, apoE2 and apoE3 which are not associated with higher risk for AD (21). Since apoE is a major brain apolipoprotein involved in lipid and cholesterol transport, it is likely that ApoE4 may alter lipid metabolism and may prevent delivery or alter metabolism or clearance of DHA or dietary lipids potentially as cholesterol esters (DHA-CE and EPA-CE) to maintain or replenish critical lipids important for neuronal function and cognition such as DHA in brain tissue and cells. Therefore having apoE4 may predispose one to development of AD due to altered DHA transport or metabolism in the brain and circulation.

It has been shown that a dietary lipid required for neuronal function, docosahexaenoic acid (22:6) (DHA) cholesterol ester (DHA-CE), is depleted in AD ventricular fluid, but not other neurodegenerative diseases (25). DHA has also been shown to be sequestered by atherosclerotic plaques (26) and may prove to be a critical link between AD and atherosclerosis. It is highly likely that a parallel phenomenon is occurring in brain and that Aβ accumulation is leading to extraction of critical dietary lipids, including DHA, from neurons could be enhanced by apoE4.

Therefore, the interaction of three variables would lead to AD, 1) amount of (reserve) DHA or other critical neuronal lipids, 2) extent of Aβ accumulation which would serve as a lipid sink in equimolar amounts to lipid, especially dietary DHA and 3) presence of the APOEε4 genotype which would alter lipid metabolism and circulation/clearance of Aβ, cholesterol esters, especially DHA-CE, and may increase deposition of Aβ preventing maintenance or replenishment of neuronal lipids to functional cellular site. Cognitive decline would be expected only after the loss of a critical mass of DHA or other important neuronal lipids or sequestration in Aβ plaques or soluble oligomers and the disruption of maintenance or replenishment of critical lipids as due to ApoE4 genotype. Targeting these interactions would allow disruption of uniquely pathological interactions therefore augmenting potential for avoiding mechanistic based side effects, which is likely to occur as the result of disrupting normal physiological function for Aβ, DHA/lipids or apoE if targeting these components of AD individually.

### 7. EXAMPLE 2: DETERMINATION OF SPECIFICITY

Experiments can be performed to further validate the Aβ/DHA/apoE interaction and to determine the specificity for binding between lipid species, different forms and lengths of Aβ peptide and different apoE isoforms. If the AD specific pathogenic Aβ42 and apoE4 alter DHA binding, data can implicate this complex in disease pathology. Studies can be executed to determine the requirement of double bonds and acyl chain length for Aβ binding. It is also possible that other commonly found Aβ species Aβ38, Aβ40, Aβ42, are specific for different acyl chain lengths with specific unsaturation requirements. Specifically, the hypotheses that Aβ38 binds arachidonic acid containing lipids (20:4); Aβ40 binds eicosapentaenoic acid (20:5) containing lipids and Aβ42 binds selectively to DHA 22:6 containing lipids, can be tested. Specificity of lipid for Aβ binding can also be determined using this assay as could the specific conformer/species of Aβ (i.e., Aβ40, Aβ42, fibril, oligomer, protofibril or monomer). Binding studies (FIGS. 1 and 2) can be used to determine which lipids form a complex with ApoE and Aβ and the extent of specificity of the ApoE:Aβ:lipid complex. Alternately, Aβ protein in form of soluble monomer, oligomer or fibril preparation (27) can be bound to reacti-bind plates and exposed to fluorescent or BODIPY-tagged lipid (i.e., DHA, 22:6) (28). The amount of bound lipid (bound to Aβ on plate) is proportional to the fluorescent signal. These studies can support the hypothesis that Aβ is a major, specific dietary lipid binding protein required for apoE mediated clearance of the lipids in the brain.

### 8. EXAMPLE 3: SMALL MOLECULE SCREEN FOR IDENTIFICATION OF EFFECTIVE BLOCKERS OF THE DHA-CE(LIPID)/Aβ/APOE INTERACTION

Small molecule libraries can be screened, e.g., in multi-well plates, for their ability to block Aβ binding to DHA-CE or disrupt the apoE:Aβ:DHA complex. Inhibitors can be identified by any of the assay platforms mentioned above, including binding lipid to the assay multi-well plate, binding Aβ to the multi-well plate or binding ApoE to the assay multi-well plate. The specificity of the interaction (lipid species, Aβ species, apoE isoform) can be determined (see Example 2, above) as the best model for the pathological complex specific for AD. Disruption of the interaction by small molecules would result in a decrease in the fluorescent signal depending on efficacy and affinity rendering this assay amenable to high-throughput screening and dose:response secondary assays.

### 9. EXAMPLE 4: TESTING Aβ:LIPID ASSOCIATED PATHOLOGY IN HUMAN BRAIN

To evaluate DHA sequestration by Aβ plaques in human brain, laser capture microdissection can be used to harvest brain cells from human autopsy brain tissue enriched with Aβ plaques or lipofuscin positive granules. Lipofuscin positive granules have been identified by original work by Alois Alzheimer as an AD-relevant pathology. They are lipid deposits which have not been characterized using modern methodologies and are likely to contain important information regarding the pathogenesis of AD (29). Only recent advances would allow microdissection of discrete areas enriched for Aβ or lipids allowing detection of regional differences which may not be apparent in lipid extract from whole brain (30,31). Either of these pathological particles (Aβ plaques or lipofuscin positive granules) may be enriched with sequestered DHA or other dietary lipid. Experiments may be performed to determine which lipids are enriched in the pathological particles while determining which lipids are de-enriched in surrounding cells/tissues lacking pathological particles and in brain cells/tissue from patients without high amyloid load.

### 10. EXAMPLE 5: LIPID RECOGNITION REGIONS

Shown below is a region which, without being bound by theory, can be the “lipid recognition” region which can coordinate with DHA unsaturated double bonds. Predicted common hydrophobic stretch with 4/8 identical amino acids is in underlined italics and were determined using Blastp (protein-protein BLAST) using scoring parameter matrix BLOSUM62 with match/mismatch scores of 1, −2; gap cost of 6 for existence and 2 for extension with conditional compositional score matrix adjustment. General parameters were automatically adjusted parameters for short input sequences with the expect threshold value set to 10 and word size allowed was 2. Cholesterol binding site of C99, identified by others, is shown in lower case bold and underlined italics with central bold capital G (124). Regions overlap at central glycine (bold capital “G”). Predicted ApoE binding region 14-17 of Aβ is depicted in non-bold capital letters (also heparin) (125). In certain non-limiting embodiments, Aβ and αS can bind ApoE in ‘hinge’ region 167-206 of ApoE amino acid sequence.

```
Aβ 42 (predicted lipid recognition region a.a. 33-40)
(SEQ ID NO: 1)
>daefrhdsgy evHHQKLvff aedvgsnkga iiGlmvggvv ia
αS 140 (predicted lipid recognition region a.a. 68-75)
(SEQ ID NO: 2)
    1 mdvfmkglsk akegvvaaae ktkqgvaeaa gktkegvlyv gsktkegvvh gvatvaektk
  61 eqvtnvggav vtgvtavaqk tvegagsiaa atgfvkkdql gkneegapqe giledmpvdp
121 dneayempse egyqdyepea
αS 126 (predicted lipid recognition region a.a. 54-61)
(SEQ ID NO: 3)
    1 mdvfmkglsk akegvvaaae ktkqgvaeaa gktkegvlyv vaektkeqvt nvggavvtgv
  61 tavaqktveg agsiaaatgf vkkdqlgkne egapqegile dmpvdpdnea yempseegyq
121 dyepea
αS 112 (predicted lipid recognition region a.a. 68-75)
(SEQ ID NO: 4)
    1 mdvfmkglsk akegvvaaae ktkqgvaeaa gktkegvlyv gsktkegvvh gvatvaektk
  61 eqvtnvggav vtgvtavaqk tvegagsiaa atgfvkkdql gkegyqdyep ea
```

### 11. EXAMPLE 6: ADMINISTRATION OF SDPC IS EFFECTIVE IN VIVO FOR PARTIAL RESCUE OF AD ASSOCIATED PHENOTYPES IN A MOUSE MODEL OF THE DISEASE TRANSGENIC FOR HUMAN APP WITH THE SWEDISH MUTATION (APPSW+)

### 11.1 Materials and Methods

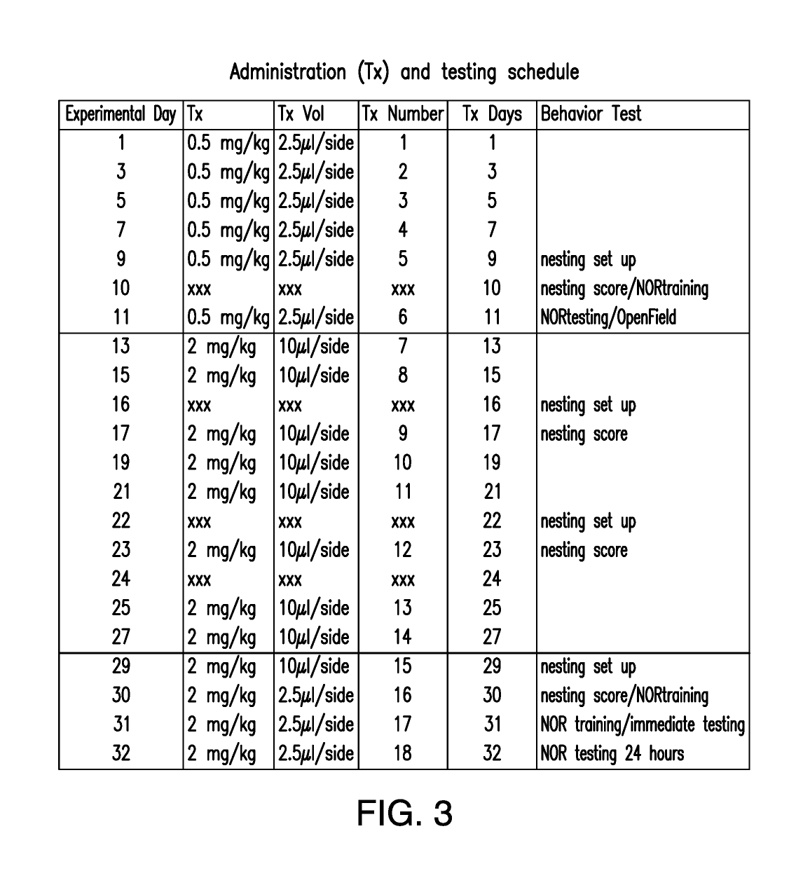

SDPC was obtained from Avanti Polar Lipids (850472C) in chloroform, dried under vacuum conditions and resuspended in 0.9% saline (0.9% sodium chloride injection, USP, NDC 0409-7983-61, Hospira) containing 0.2% (weight:volume) methyl cellulose (average Mn 40,000, viscosity: 400 cP, CAS 9004-67-5, Sigma-Aldrich 274429) to aid in solubilization. A control solution of 0.9% saline containing 0.2% methyl cellulose was prepared at the same time without SDPC. A concentration of 3 mg/ml was used for doses 1-15 and 12 mg/ml was used for doses 16-18 (FIG. 3). Brief (3-5 minutes) bath sonication was used to improve solubility of 12 mg/ml concentration.

Mice were treated for 10 days at a low dose of SDPC intranasally administered 2.5 uL each nostril (5 μL total dose) for 0.5 mg/kg every other day assuming average mouse weight of 30 g (0.03 kg) (FIG. 3). After 10 days, dose was escalated to 2 mg/kg every other day for an additional 19 days (total treatment time 32 days). Doses 16-18 were administered daily.

### 11.2 Results

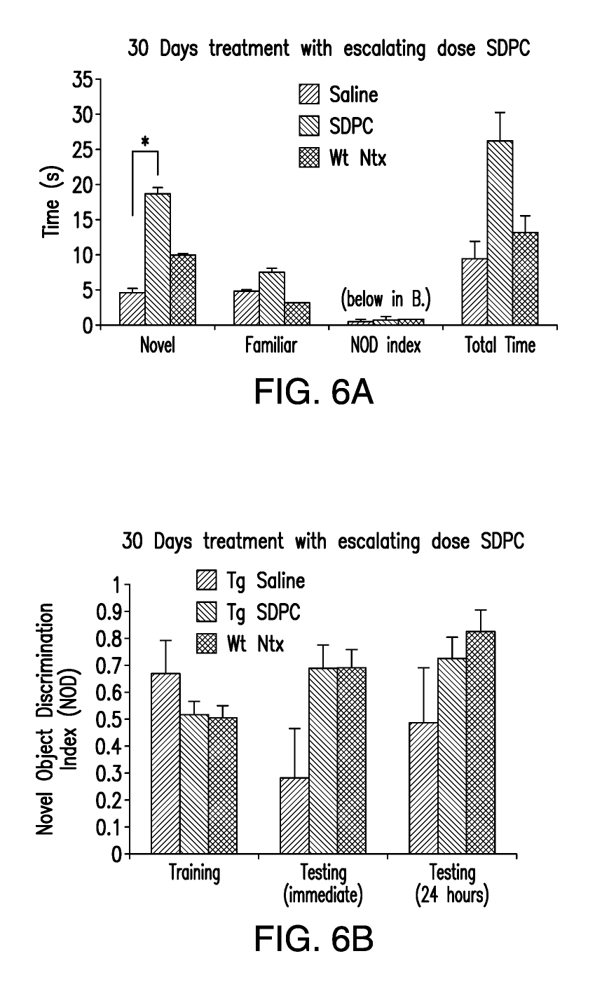

APPsw+mice show behavioral deficits such as impaired novel object recognition (NOR) (118) (FIGS. 4 and 6). After 10 days intranasal treatment with (SDPC), there is a trend toward improvement for nesting behavior and total activity level (FIGS. 4A and 4B) and a trend toward improved exploratory behavior as well as continued improvement of activity level (FIGS. 4C and 4D).

Non-invasive behavioral testing using novel object recognition (118) and Nesting behavior (114) were used to assess behavioral function. Deficits were expected in APPsw+(Tg) mice and compared to wild type littermates of the same age (13-14 months). APPsw+mice were treated (Tx) with either control solution of 0.9% saline containing 0.2% methyl cellulose [Saline] or SDPC in 0.9% saline containing 0.2% methyl cellulose [SDPC]. After 10 days treatment at low dose, non-significant trend for improvement in nesting behavior was observed (FIG. 4A), as well as a non-significant trend for improvement in activities common to wild type animals such as wall rearing and free rearing (FIG. 4B). Significant changes were seen using Student's t-test comparing SDPC and wild type (non-treated) [WT Ntx] and comparing saline and WT Ntx, but not when comparing SDPC and saline groups likely due to small sample size (n=4-5). Time spent with each of two identical objects, left object (L obj) and object on the right (R obj) as well as total time (L obj+R obj) during NOR training is shown indicating a non-significant trend toward exploration activity when APPsw+mice are treated with SDPC (FIG. 4C). After 24 hours, mice were tested for NOR discrimination by replacing one object with a novel object, however, no discrimination was found. Activity in the open field test following NOR testing indicated mice positive for APPsw+show reduced activity, but when treated with SDPC, activity level is restored to the level of wild type non-treated mice (WTNtx) but does not reach significance likely due to the small sample size [Saline treated APPsw+(control) (n=4); SDPC treated APPsw+(n=5) and wild type receiving no treatment (n=4)].

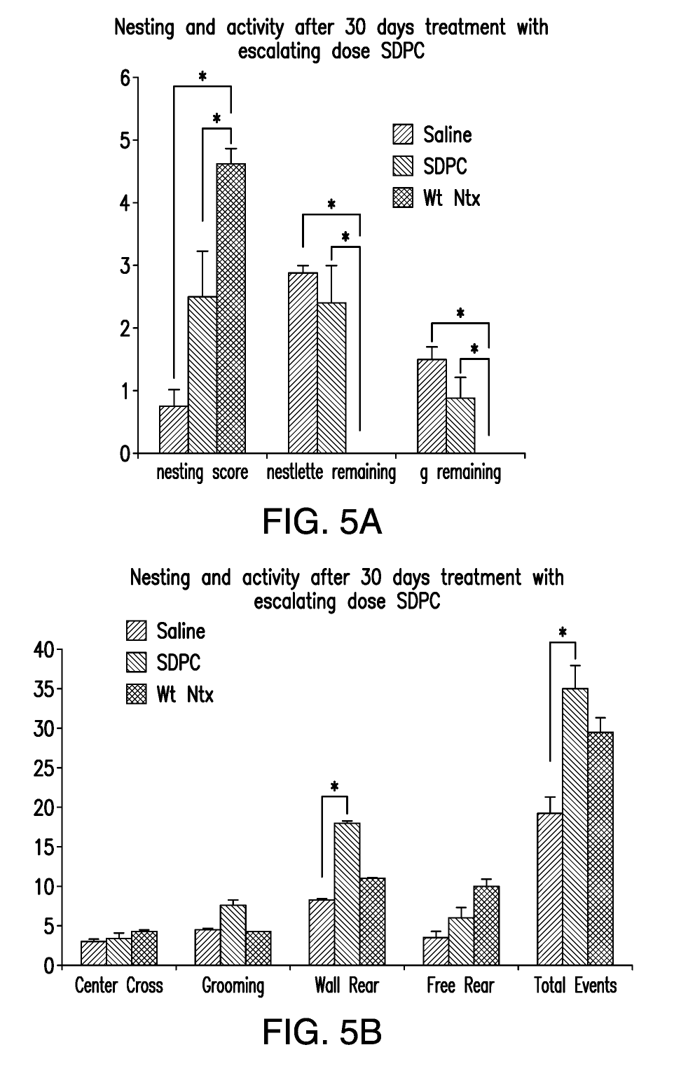

Intranasal administration of SDPC for 30 days with escalated dose (FIG. 3) resulted in improved activity level including a significant improvement in number of wall rears and total activity events between saline (control) treated APPsw+ and SDPC treated APPsw+mice (FIG. 5). A significant amelioration of NOR deficits is shown due to the increased time APPsw+mice spend with a novel object (FIG. 6). Though significant changes were not observed for novel object discrimination index (NOD index) likely due to small sample size [Saline treated APPsw+(control) (n=4); SDPC treated APPsw+(n=5) and wild type receiving no treatment (n=4)]. A trend for amelioration of NOD defect is apparent with SDPC treatment.

### 11.3 Discussion

The present mouse model of AD can also be used to perform a full dose response curve study. Additionally, further studies exploring the specificity for DHA and EPA components of different lipid species, such as phosphatidylcholine, phosphatidylethanoloamine, cholesterol esters, phospholipids, plasmalogens, triglycerides, gangliosides, and celebrosides for binding affinity to Aβ species including Aβ38, Aβ40, Aβ42 as well as different oligomeric states using the assay described above can guide precise formulation of lipid for treatment.

Moreover, full ADME toxicology studies are also proposed, though toxicity due to phosphatidylcholine or other lipids composed of DHA is highly unlikely since this is a naturally occurring component of eggs. However, egg allergy should be considered.

Lastly, other mouse model that can be used to assess the above-mentioned parameters include secondary models of AD (such as J20), as well as mouse models of Down Syndrome (such as Ts65Dn or Ts1Cje).

### 12. REFERENCES

1. Tu S, Okamoto S, Lipton S A, Xu H. (2014) Oligomeric Aβ-induced synaptic dysfunction in Alzheimer's disease. Mol Neurodegener. November 14; 9:48. doi: 10.1186/1750-1326-9-48.
2. Ferreira S T, Lourenco M V, Oliveira M M, De Felice F G. (2015) Soluble amyloid-β oligomers as synaptotoxins leading to cognitive impairment in Alzheimer's disease. Front Cell Neurosci. May 26; 9:191.
3. Bohm C, Chen F, Sevalle J, Qamar S, Dodd R, Li Y, Schmitt-Ulms G, Fraser P E, St George-Hyslop P H. (2015) Current and future implications of basic and translational research on amyloid-β peptide production and removal pathways. Mol Cell Neurosci. May; 66(Pt A): 3-11.
4. Joffre C, Nadjar A, Lebbadi M, Calon F, Laye S. (2014). n−3 LCPUFA improves cognition: the young, the old and the sick. Prostaglandins Leukot Essent Fatty Acids. July-August; 91(1-2): 1-20.
5. Hashimoto M, Hossain S, Yamasaki H, Yazawa K, Masumura S. (1999) Effects of eicosapentaenoic acid and docosahexaenoic acid on plasma membrane fluidity of aortic endothelial cells. Lipids. 1999 December; 34(12):1297-304.
6. Janssen C I, Kiliaan A J. (2014) Long-chain polyunsaturated fatty acids (LCPUFA) from genesis to senescence: the influence of LCPUFA on neural development, aging, and neurodegeneration. Prog Lipid Res. January; 53:1-17.
7. Hashimoto M, Hossain S, Katakura M, Al Mamun A, Shido 0. (2015) The binding of Aβ1-42 to lipid rafts of RBC is enhanced by dietary docosahexaenoic acid in rats: Implicates to Alzheimer's disease. Biochim Biophys Acta. June; 1848(6): 1402-9.
8. Jiao J, Li Q, Chu J, Zeng W, Yang M, Zhu S. (2014) Effect of n−3 PUFA supplementation on cognitive function throughout the life span from infancy to old age: a systematic review and meta-analysis of randomized controlled trials. Am J Clin Nutr. December; 100(6): 1422-36.
9. Song Y, Kenworthy A K, Sanders C R. (2014) Cholesterol as a co-solvent and a ligand for membrane proteins. Protein Sci. January; 23(1):1-22.
10. Xiong J, Roach C A, Oshokoya 00, Schroell R P, Yakubu R A, Eagleburger M K, Cooley J W, Jiji R D. (2014) Role of bilayer characteristics on the structural fate of a13(1-40) and 43(25-40). Biochemistry. May 13; 53(18):3004-11.
11. Lockhart C, Klimov D K. (2014) Alzheimer's Aβ10-40 peptide binds and penetrates DMPC bilayer: an isobaric-isothermal replica exchange molecular dynamics study. J Phys Chem B. Mar. 13; 118(10):2638-48.
12. Yates E A, Owens S L, Lynch M F, Cucco E M, Umbaugh C S, Legleiter J. (2013) Specific domains of Aβ facilitate aggregation on and association with lipid bilayers. J Mol Biol. June 12; 425(11):1915-33.
13. Sasahara K, Morigaki K, Shinya K. (2013) Effects of membrane interaction and aggregation of amyloid β-peptide on lipid mobility and membrane domain structure. Phys Chem Chem Phys. June 21; 15(23):8929-39.
14. Vitiello G, Di Marino S, D'Ursi A M, D'Errico G. (2013) Omega-3 fatty acids regulate the interaction of the Alzheimer's a13(25-35) peptide with lipid membranes. Langmuir. November 19; 29(46):14239-45.
15. Manna M, Mukhopadhyay C. (2013) Binding, conformational transition and dimerization of amyloid-0 peptide on GM1-containing ternary membrane: insights from molecular dynamics simulation. PLoS One. August 9; 8(8):e71308.
16. Fantini J, Yahi N, Garmy N. (2013) Cholesterol accelerates the binding of Alzheimer's β-amyloid peptide to ganglioside GM1 through a universal hydrogen-bond-dependent sterol tuning of glycolipid conformation. Front Physiol. June 10; 4:120.
17. Barulli D, Stern Y. Efficiency, capacity, compensation, maintenance, plasticity: emerging concepts in cognitive reserve. Trends Cogn Sci. 2013 October; 17(10):502-9.
18. Pelletier A, Barul C, Féart C, Helmer C, Bernard C, Periot O, Dilharreguy B, Dartigues J F, Allard M, Barberger-Gateau P, Catheline G, Samieri C. Mediterranean diet and preserved brain structural connectivity in older subjects. Alzheimers Dement. 2015 September; 11(9):1023-31.
19. Safouris A, Tsivgoulis G, Sergentanis T N, Psaltopoulou T. Mediterranean Diet and risk of Dementia. Curr Alzheimer Res. 2015; 12(8):736-44.
20. Rosenblum W I, Why Alzheimer trials fail: removing soluble oligomeric beta amyloid is essential, inconsistent, and difficult. Neurobiology of Aging. Volume 35, Issue 5, May 2014, Pages 969-974.
21. Castellano J M, Kim J, Stewart F R, Jiang H, DeMattos R B, Patterson B W, Fagan A M, Morris J C, Mawuenyega K G, Cruchaga C, et al. 2011. Human apoE isoforms differentially regulate brain amyloid-0 peptide clearance. Sci Transl Med 3: 89ra57.
22. Kim J, Basak J M, Holtzman D M. 2009. The role of apolipoprotein E in Alzheimer's disease. Neuron 63: 287-303.
23. Dorey E, Chang N, Liu Q Y, Yang Z, Zhang W. (2014) Apolipoprotein E, amyloid-beta, and neuroinflammation in Alzheimer's disease. Neurosci Bull. April; 30(2):317-30.
24. Poirier J, Miron J, Picard C, Gormley P, Théroux L, Breitner J, Dea D. (2014) Apolipoprotein E and lipid homeostasis in the etiology and treatment of sporadic Alzheimer's disease. Neurobiol Aging. September; 35 Suppl 2:S3-10.
25. Montine, T. J. Montine K. S.and Swift. L. L. (1997) Central nervous system lipoproteins in Alzheimer's disease.. Americal Journal of Pathology, Vol 151, No 6:1571-1575.
26. Rapp J H, Conner W E, Lin D S and Porter J M. (1991) Dietary Eicosapentaenoic Acid and Docosahexaenoic Acid From Fish Oil, Their Incorporation Into Advanced Human Atherosclerotic Plaques Arteriosclerosis and Thrombosis Vol 11, No 4: 903-911.
27. Dahlgren K N, Manelli A M, Stine W B Jr, Baker L K, Krafft G A, LaDu M J. Oligomeric and fibrillar species of amyloid-beta peptides differentially affect neuronal viability. J Biol Chem. 2002 Aug. 30; 277(35):32046-53.
28. Teague H, Ross R, Harris M, Mitchell D C, Shaikh S R. DHA-fluorescent probe is sensitive to membrane order and reveals molecular adaptation of DHA in ordered lipid microdomains. J Nutr Biochem. 2013 January; 24(1):188-95.
29. Foley P. (2010) Lipids in Alzheimer's disease: A century-old story. Biochim Biophys Acta. August; 1801(8):750-3.
30. Datta S, Malhotra L, Dickerson R, Chaffee S, Sen C K, Roy S. (2015) Laser capture microdissection: Big data from small samples. Histol Histopathol. 2015 Apr. 20:11622.
31. Longuespée R, Fléron M, Pottier C, Quesada-Calvo F, Meuwis M A, Baiwir D, Smargiasso N, Mazzucchelli G, De Pauw-Gillet M C, Delvenne P, De Pauw E. (2014) Tissue proteomics for the next decade? Towards a molecular dimension in histology. OMICS. 2014 September; 18(9):539-52.
32. Antalis C J, Arnold T, Lee B, Buhman K K, Siddiqui R A. (2009) Docosahexaenoic acid is a substrate for ACAT1 and inhibits cholesteryl ester formation from oleic acid in MCF-10A cells. Prostaglandins Leukot Essent Fatty Acids. February-March; 80(2-3): 165-71.
33. Dyall S C. (2015) Long-chain omega-3 fatty acids and the brain: a review of the independent and shared effects of EPA, DPA and DHA. Front Aging Neurosci. Apr. 21; 7:52.
34. Frits A. J. Muskiet, M. Rebecca Fokkema, Anne Schaafsma, E. Rudy Boersma and Michael A. Crawford. (2004) American Society for Nutritional Sciences. J. Nutr. 134:183-186, 2004.
35. Fouquet M, Besson F L, Gonneaud J, La Joie R, Chételat G. (2014) Imaging brain effects of APOE4 in cognitively normal individuals across the lifespan. Neuropsychol Rev. September; 24(3):290-9.
36. Grimm M O, Zimmer V C, Lehmann J, Grimm H S, Hartmann T. (2013). The impact of cholesterol, DHA, and sphingolipids on Alzheimer's disease. Biomed Res Int.; 2013:814390.
37. Izem Ll, Morton R E. (2007) Possible role for intracellular cholesteryl ester transfer protein in adipocyte lipid metabolism and storage. J Biol Chem. 2007 July 27; 282(30):21856-65.
38. Lane-Donovan C, Philips G T, Herz J. (2014) More than cholesterol transporters: lipoprotein receptors in CNS function and neurodegeneration. Neuron. 2014 Aug. 20; 83(4):771-87.
39. Lesne S E. (2014) Toxic oligomer species of amyloid-β in Alzheimer's disease, a timing issue. Swiss Med Wkly. 2014 Nov. 6; 144:w14021.
40. Li H, Dhanasekaran P, Alexander E T, Rader D J, Phillips M C, Lund-Katz S. (2013) Molecular mechanisms responsible for the differential effects of apoE3 and apoE4 on plasma lipoprotein-cholesterol levels. Arterioscler Thromb Vasc Biol. 2013 April; 33(4):687-93.
41. Morris M C, Tangney C C. (2014) Dietary fat composition and dementia risk. Neurobiol Aging. 2014 September; 35 Suppl 2:S59-64.
42. Redgrave T G. (1970) Formation of cholesteryl ester-rich particulate lipid during metabolism of chylomicrons. J Clin Invest. 1970 March; 49(3):465-71.
43. Xu Z, Riediger N, Innis S, Moghadasian M H, (2007). Fish oil significantly alters fatty acid profiles in various lipid fractions but not atherogenesis in apo E-K O mice. Eur J Nut 46:103-110.
44. Yue S, Li J, Lee S Y, Lee H J, Shao T, Song B, Cheng L, Masterson T A, Liu X, Ratliff T L, Cheng J X (2014) Cholesteryl ester accumulation induced by PTEN loss and PI3K/AKT activation underlies human prostate cancer aggressiveness. Cell Metab. 2014 Mar. 4; 19(3):393-406.
45. Dubnovitsky A, Sandberg A, Rahman M M, Benilova I, Lendel C, Hard T. (2013) Amyloid-β protofibrils: size, morphology and synaptotoxicity of an engineered mimic. PLoS One. July 2; 8(7):e66101.
46. Hanson A J, Bayer-Carter J L, Green P S, Montine T J, Wilkinson C W, Baker L D, Watson G S, Bonner L M, Callaghan M, Leverenz J B, Tsai E, Postupna N, Zhang J, Lampe J, Craft S. (2013) Effect of apolipoprotein E genotype and diet on apolipoprotein E lipidation and amyloid peptides: randomized clinical trial. JAMA Neurol. 2013 August; 70(8):972-80.
47. Kuszczyk M A, Sanchez S, Pankiewicz J, Kim J, Duszczyk M, Guridi M, Asuni A A, SullivanPM, Holtzman D M, Sadowski M J. Blocking the interaction between apolipoprotein E and Aβ reduces intraneuronal accumulation of Aβ and inhibits synaptic degeneration. Am J Pathol. 2013 May; 182(5):1750-68.
48. Liu S, Breitbart A, Sun Y, Mehta P D, Boutajangout A, Scholtzova H, Wisniewski T. (2014) Blocking the apolipoprotein E/amyloid β interaction in triple transgenic mice ameliorates Alzheimer's disease related amyloid β and tau pathology. J Neurochem. 2014 February; 128(4):577-91.
49. Salvati E, Re F, Sesana S, Cambianica I, Sancini G, Masserini M, Gregori M. (2013) Liposomes functionalized to overcome the blood-brain barrier and to target amyloid-β peptide: the chemical design affects the permeability across an in vitro model. Int J Nanomedicine.; 8:1749-58.
50. Song Q, Huang M, Yao L, Wang X, Gu X, Chen J, Chen J, Huang J, Hu Q, Kang T, Rong Z, Qi H, Zheng G, Chen H, Gao X. (2014). Lipoprotein-based nanoparticles rescue the memory loss of mice with Alzheimer's disease by accelerating the clearance of amyloid beta. ACS Nano. Mar. 25; 8(3):2345-59.
51. Tai L M, Mehra S, Shete V, Estus S, Rebeck G W, Bu G, LaDu M J. (2014) Soluble apoE/Aβ complex: mechanism and therapeutic target for APOE4-induced A D risk. Mol Neurodegener. 2014 Jan. 4; 9:2.
52. Torres M, Price S L, Fiol-Deroque M A, Marcilla-Etxenike A, Ahyayauch H, Barceló-Coblijn G, Teres S, Katsouri L, Ordinas M, Lopez D J, Ibarguren M, Goñi F M, Busquets X, Vitorica J, Sastre M, Escriba P V. (2014) Membrane lipid modifications and therapeutic effects mediated by hydroxydocosahexaenoic acid on Alzheimer's disease. Biochim Biophys Acta. June; 1838(6):1680-92.
53. Datta S, Malhotra L, Dickerson R, Chaffee S, Sen C K, Roy S. (2015) Laser capture microdissection: Big data from small samples. Histol Histopathol. 2015 Apr. 20:11622.
54. Diaz O, Berquand A, Dubois M, Di Agostino S, Sette C, Bourgoin S, Lagarde M, Nemoz G, Prigent A F (2002) The mechanism of docosahexaenoic acid-induced phospholipase D activation in human lymphocytes involves exclusion of the enzyme from lipid rafts. J Biol Chem 277:39368-39378.
55. Farkas T, Kitajka K, Fodor E, Csengeri I, Landes E, Yeo Y K, Krasznai Z, Halver J E. (2000) Docosahexaenoic acid-containing phospholipid molecular species in brains of vertebrates. Proc Natl Acad Sci USA 97:6362-6366.
56. Foley P. (2010) Lipids in Alzheimer's disease: A century-old story. Biochim Biophys Acta. August; 1801(8):750-3.
57. Gonzalez-Periz A; Planaguma A; Gronert K; Miguel R; Lopez-Parra M; Titos E; Horrillo R; Ferre N; Deulofeu R; Arroyo V; Rodes J; Claria J (2007) Docosahexaenoic acid (DHA) blunts liver injury by conversion to protective lipid mediators: protectin D1 and 17S-hydroxy-DHA. The Faseb Journal: Official Publication of the Federation of American Societies for Experimental Biology 14:2537-2539.
58. Hashimoto M, Hossain S, Yamasaki H, Yazawa K, Masumura S. (1999) Effects of eicosapentaenoic acid and docosahexaenoic acid on plasma membrane fluidity of aortic endothelial cells. Lipids 34:1297-1304.
59. Lee H, Kim Y, Park A, Nam J M. (2014) Amyloid-β aggregation with gold nanoparticles on brain lipid bilayer. Small. 2014 May 14; 10(9):1779-89.
60. Ni R, Gillberg P G, Bergfors A, Marutle A, Nordberg A. (2013) Amyloid tracers detect multiple binding sites in Alzheimer's disease brain tissue. Brain. 2013 July; 136(Pt 7):2217-27.
61. Torres M, Price S L, Fiol-Deroque M A, Marcilla-Etxenike A, Ahyayauch H, Barceló-Coblijn G, Teres S, Katsouri L, Ordinas M, Lopez D J, Ibarguren M, Goñi F M, Busquets X, Vitorica J, Sastre M, Escriba P V. (2014) Membrane lipid modifications and therapeutic effects mediated by hydroxydocosahexaenoic acid on Alzheimer's disease. Biochim Biophys Acta. June; 1838(6):1680-92.
62. Yahi N, Fantini J. (2014) Deciphering the glycolipid code of Alzheimer's and Parkinson's amyloid proteins allowed the creation of a universal ganglioside-binding peptide. PLoS One. August 20; 9(8):e104751.
63. Yang Y, Keene C D, Peskind E R, Galasko D R, Hu S C, Cudaback E, Wilson A M, Li G, Yu C E, Montine K S, Zhang J, Baird G S, Hyman B T, Montine T J. (2015) Cerebrospinal Fluid Particles in Alzheimer Disease and Parkinson Disease. J Neuropathol Exp Neurol. July; 74(7):672-87.
64. Al Asmari A K, Ullah Z, Tariq M, Fatani A. Preparation, characterization, and in vivo evaluation of intranasally administered liposomal formulation of donepezil. Drug Des Devel Ther. 2016 2016; 10: 205-215.
65. Anttila, M., et al., Bioavailability of dexmedetomidine after extravascular doses in healthy subjects. Br. J Clin Pharmacol, 2003. 56: p. 691-693.
66. Banks, W. A., M. J. During, and M. L. Niehoff, Brain uptake of the glucagon-like peptide-1 antagonist exendin(9-39) after intranasal administration. J Pharmacol Exp Ther, 2004. 309(2): p. 469-75.
67. Bartels T, Choi J G, Selkoe D J. a-Synuclein occurs physiologically as a helically folded tetramer that resists aggregation. Nature. 2011 Aug. 14; 477(7362):107-10.
68. Bohm C, Chen F, Sevalle J, Qamar S, Dodd R, Li Y, Schmitt-Ulms G, Fraser P E, St George-Hyslop P H. (2015) Current and future implications of basic and translational research on amyloid-β peptide production and removal pathways. Mol Cell Neurosci. May; 66(Pt A): 3-11.
69. Carstea E D, Morris J A, Coleman K G, Loftus S K, Zhang D, Cummings C, Gu J, Rosenfeld M A, Pavan W J, Krizman D B, Nagle J, Polymeropoulos M H, Sturley S L, Ioannou Y A, Higgins M E, Comly M, Cooney A, Brown A, Kaneski C R, Blanchette-Mackie E J, Dwyer N K, Neufeld E B, Chang T Y, Liscum L, Strauss J F 3rd, Ohno K, Zeigler M, Carmi R, Sokol J, Markie D, O'Neill R R, van Diggelen O P, Elleder M, Patterson M C, Brady R O, Vanier M T, Pentchev P G, Tagle D A. Niemann-Pick C1 disease gene: homology to mediators of cholesterol homeostasis. Science. 1997 Jul. 11; 277(5323):228-31.
70. Carver J D, Benford V J, Han B, Cantor A B. The relationship between age and the fatty acid composition of cerebral cortex and erythrocytes in human subjects. Brain Res Bull. 2001 Sep. 15; 56(2):79-85.
71. Conway K A, Lee S J, Rochet J C, Ding T T, Williamson R E, Lansbury P T Jr. Acceleration of oligomerization, not fibrillization, is a shared property of both alpha-synuclein mutations linked to early-onset Parkinson's disease: implications for pathogenesis and therapy. Proc Natl Acad Sci USA. 2000 Jan. 18; 97(2):571-6.
72. Cunnane S C, Chouinard-Watkins R, Castellano C A, Barberger-Gateau P. Docosahexaenoic acid homeostasis, brain aging and Alzheimer's disease: Can we reconcile the evidence? Prostaglandins Leukot Essent Fatty Acids. 2013 January; 88(1):61-70.
73. Dale, O., R. Hjortkjaer, and E. D. Kharasch, Nasal administration of opioids for pain management in adults. Acta Anaesthesiol Scand, 2002. 46(7): p. 759-70.
74. Davidson W S, Jonas A, Clayton D F, George J M. Stabilization of alpha-synuclein secondary structure upon binding to synthetic membranes. J Biol Chem. 1998 Apr. 17; 273(16):9443-9.
75. Davis A A, Andruska K M, Benitez B A, Racette B A, Perlmutter J S, Cruchaga C. Variants in GBA, SNCA, and MAPT influence Parkinson disease risk, age at onset, and progression. Neurobiol Aging. 2016 January; 37:209.e1-7.
76. Fernandez C O, Hoyer W, Zweckstetter M, Jares-Erijman E A, Subramaniam V, Griesinger C, Jovin T M. NMR of alpha-synuclein-polyamine complexes elucidates the mechanism and kinetics of induced aggregation. EMBO J. 2004 May 19; 23(10):2039-46.
77. Golabek A A, Soto C, Vogel T, Wisniewski T. The interaction between apolipoprotein E and Alzheimer's amyloid beta-peptide is dependent on beta-peptide conformation. J. Biol. Chem., 271 (1996), pp. 10602-10606.
78. Gurzell E A, Wiesinger J A, Morkam C, Hemmrich S, Harris W S, Fenton J I. Is the omega-3 index a valid marker of intestinal membrane phospholipid EPA+DHA content? Prostaglandins Leukot Essent Fatty Acids. 2014 September; 91(3):87-96.
79. Hardy, J. G., S. W. Lee, and C. G. Wilson, Intranasal drug delivery by spray and drops. J Pharm Pharmacol, 1985. 37(5): p. 294-7.
80. Hartley D, et al. (2015) Down syndrome and Alzheimer's disease: Common pathways, common goals. Alzheimers Dement. June; 11(6):700-9.
81. Hatch, T. F., Distribution and deposition of the inhaled particles in respiratory tract. Bact Rev, 1961. 25: p. 237.
82. Henry, R. J., et al., A pharmacokinetic study of midazolam in dogs: nasal drop vs. atomizer administration. Pediatr Dent, 1998. 20(5): p. 321-6.
83. Khalil, S., H. Vije, and S. Kee, A pediatric trial comparing midazolam/syrpalta mixture with premixed midazolam syrup (Roche). Paediatr Anaesth, 2003. 13: p. 205-209.
84. Kojro E, Gimpl G, Lammich S, Marz W, Fahrenholz F. Low cholesterol stimulates the nonamyloidogenic pathway by its effect on the alpha-secretase ADAM 10. Proc Natl Acad Sci USA. 2001 May 8; 98(10):5815-20.
85. Kumar A, Bullard R L, Patel P, Paslay L C, Singh D, Bienkiewicz E A, Morgan S E, Rangachari V. Non-Esterified Fatty Acids Generate Distinct Low-Molecular Weight Amyloid-β (Aβ42) Oligomers along Pathway Different from Fibril Formation. PLoS One. 2011; 6(4): e18759.
86. Kumar A, Paslay L C, Lyons D, Morgan S E, Correia J J, Rangachari V. Specific Soluble Oligomers of Amyloid-β Peptide Undergo Replication and Form Non-fibrillar Aggregates in Interfacial Environments J Biol Chem. 2012 June 15; 287(25): 21253-21264.
87. Lashuel H A, Overk C R, Oueslati A, Masliah E. The many faces of a-synuclein: from structure and toxicity to therapeutic target. Nat Rev Neurosci. 2013 January; 14(1):38-48.
88. Malnar M, Hecimovic S, Mattsson N, Zetterberg H. Bidirectional links between Alzheimer's disease and Niemann-Pick type C disease. Neurobiol Dis. 2014 December; 72 Pt A:37-47.
89. McCann, H; Stevens, C. H.; Cartwright, H; Halliday, G. M. (2014). “A-Synucleinopathy phenotypes”. Parkinsonism & Related Disorders. 20 Suppl 1: S62-7.
90. Mygind, N. and S. Vesterhauge, Aerosol distribution in the nose. Rhinology, 1978. 16(2): p. 79-88.
91. Mygind, N., Nasal Allergy, 2nd edition. Blackwell, Oxford, England, 1979: p. 257-270.
92. Nemani V M, Lu W, Berge V, Nakamura K, Onoa B, Lee M K, Chaudhry F A, Nicoll R A, Edwards R H. Increased expression of alpha-synuclein reduces neurotransmitter release by inhibiting synaptic vesicle reclustering after endocytosis. Neuron. 2010 Jan. 14; 65(1):66-79.
93. Nordgren T M, Friemel T D, Heires A J, Poole J A, Wyatt T A, Romberger D J. The omega-3 fatty acid docosahexaenoic acid attenuates organic dust-induced airway inflammation. Nutrients. 2014 Nov. 27; 6(12):5434-52.
94. Okamura N, Kiuchi S, Tamba M, Kashima T, Hiramoto S, Baba T, Dacheux F, Dacheux J L, Sugita Y, Jin Y Z. A porcine homolog of the major secretory protein of human epididymis, HE1, specifically binds cholesterol. Biochim Biophys Acta. 1999 Jun. 10; 1438(3):377-87.
95. Sakane, T., et al., Transport of cephalexin to the cerebrospinal fluid directly from the nasal cavity. J Pharm Pharmacol, 1991. 43(6): p. 449-51.
96. Schupf N, Zigman W B, Tang M X, Pang D, Mayeux R, Mehta P, Silverman W. (2010). Change in plasma A B peptides and onset of dementia in adults with Down syndrome. Neurology. November 2; 75(18):1639-44.
97. Segrest J P, Jones M K, De Loof H, Brouillette C G, Venkatachalapathi Y V, Anantharamaiah G M. The amphipathic helix in the exchangeable apolipoproteins: a review of secondary structure and function. J Lipid Res. 1992 February; 33(2):141-66.
98. Serth J, Lautwein A, Frech M, Wittinghofer A, Pingoud A. The inhibition of the GTPase activating protein-Ha-ras interaction by acidic lipids is due to physical association of the C-terminal domain of the GTPase activating protein with micellar structures. EMBO J. 1991 June; 10(6):1325-30.
99. Shi Z, Sachs J N, Rhoades E, Baumgart T. Biophysics of a-synuclein induced membrane remodelling. Phys Chem Chem Phys. 2015 Jun. 28; 17(24):15561-8.
100. Shinde R L, Bharkad G P, Devarajan P V. Intranasal microemulsion for targeted nose to brain delivery in neurocysticercosis: Role of docosahexaenoic acid. Eur J Pharm Biopharm. 2015 October; 96:363-79.
101. Stuart, B. O., Deposition of inhaled aerosols. Arch Intern Med, 1973. 131(1): p. 60-73.
102. Surguchov A., Intracellular Dynamics of Synucleins: “Here, There and Everywhere”. Int Rev Cell Mol Biol. 2015; 320:103-69.
103. Tsuang D, Leverenz J B, Lopez O L, Hamilton R L, Bennett D A, Schneider J A, Buchman A S, Larson E B, Crane P K, Kaye J A, Kramer P, Woltjer R, Trojanowski J Q, Weintraub D, Chen-Plotkin A S, Irwin D J, Rick J, Schellenberg G D, Watson G S, Kukull W, Nelson P T, Jicha G A, Neltner J H, Galasko D, Masliah E, Quinn J F, Chung K A, Yearout D, Mata I F, Wan J Y, Edwards K L, Montine T J, Zabetian C P. APOE c4 increases risk for dementia in pure synucleinopathies. JAMA Neurol. 2013 February; 70(2):223-8.
104. Ulmer T S, Bax A, Cole N B, Nussbaum R L Structure and dynamics of micelle-bound human alpha-synuclein. J Biol Chem. 2005 Mar. 11; 280(10):9595-603.
105. van Maarschalkerweerd A, Vetri V, Vestergaard B. Cholesterol facilitates interactions between a-synuclein oligomers and charge-neutral membranes. FEBS Lett. 2015 Sep. 14; 589(19 Pt B):2661-7.
106. von Arnim C A, von Einem B, Weber P, Wagner M, Schwanzar D, Spoelgen R, Strauss W L, Schneckenburger H. Impact of cholesterol level upon APP and BACE proximity and APP cleavage. Biochem Biophys Res Commun. 2008 May 30; 370(2):207-12.
107. Vekrellis K, Xilouri M, Emmanouilidou E, Rideout H J, Stefanis L. Pathological roles of a-synuclein in neurological disorders. Lancet Neurol. 2011 November; 10(11):1015-25.
108. Wales P, Pinho R, Lázaro D F, Outeiro T F. Limelight on alpha-synuclein: pathological and mechanistic implications in neurodegeneration. J Parkinsons Dis. 2013; 3(4):415-59.
109. Wang W, Perovic I, Chittuluru J, Kaganovich A, Nguyen L T, Liao J, Auclair J R, Johnson D, Landeru A, Simorellis A K, Ju S, Cookson M R, Asturias F J, Agar J N, Webb B N, Kang C, Ringe D, Petsko G A, Pochapsky T C, Hoang Q Q. A soluble a-synuclein construct forms a dynamic tetramer. Proc Natl Acad Sci USA. 2011 Oct. 25; 108(43): 17797-802.
110. Westin, et al., Direct nose-to-brain transfer of morphine after nasal administration to rats. Pharm Res, 2006. 23(3): p. 565-72.
111. Yuen, V. M., et al., A comparison of intranasal dexmedetomidine and oral midazolam for premedication in pediatric anesthesia: a double-blinded randomized controlled trial. Anesth Analg, 2008. 106(6): p. 1715-21.
112. Compositions for nasal delivery, Publication number WO2007043057A2, Application number PCT/IL2006/001187.
113. Bahadur S, Pathak K. Physicochemical and physiological considerations for efficient nose-to-brain targeting. Expert Opin Drug Deliv. 2012 January; 9(1):19-31.
114. Deacon R M, Assessing nest building in mice. Nat Protoc. 2006; 1(3):1117-9.
115. Demeester N, Castro G, Desrumaux C, De Geitere C, Fruchart J C, Santens P, Mulleners E, Engelborghs S, De Deyn P P, Vandekerckhove J, Rosseneu M, Labeur C. Characterization and functional studies of lipoproteins, lipid transfer proteins, and lecithin:cholesterol acyltransferase in CSF of normal individuals and patients with Alzheimer's disease. J Lipid Res. 2000 June; 41(6):963-74.
116. Hanson L R, Frey W H 2nd. Intranasal delivery bypasses the blood-brain barrier to target therapeutic agents to the central nervous system and treat neurodegenerative disease. BMC Neurosci. 2008 Dec. 10; 9 Suppl 3:S5.
117. Lukiw W J, Bazan N G. Docosahexaenoic acid and the aging brain. J Nutr. 2008 December; 138(12):2510-4.
118. McIntire L B, Berman D E, Myaeng J, Staniszewski A, Arancio O, Di Paolo G, Kim T W. Reduction of synaptojanin 1 ameliorates synaptic and behavioral impairments in a mouse model of Alzheimer's disease. J Neurosci. 2012 Oct. 31; 32(44):15271-6.
119. Mi W, van Wijk N, Cansev M, Sijben J W, Kamphuis P J Nutritional approaches in the risk reduction and management of Alzheimer's disease. Nutrition. 2013 September; 29(9): 1080-9.
120. van Wijk Nl, Broersen L M, de Wilde M C, Hageman R J, Groenendijk M, Sijben J W, Kamphuis P J. Targeting synaptic dysfunction in Alzheimer's disease by administering a specific nutrient combination. J Alzheimers Dis. 2014; 38(3):459-79.
121. Vitali C, Wellington C L, Calabresi L. HDL and cholesterol handling in the brain. Cardiovasc Res. 2014 Aug. 1; 103(3):405-13.
122. Vuletic S, Jin L W, Marcovina S M, Peskind E R, Moller T, Albers J J. Widespread distribution of PLTP in human CNS: evidence for PLTP synthesis by glia and neurons, and increased levels in Alzheimer's disease. J Lipid Res. 2003 Jun.; 44(6): 1113-23.
123. Djupesland P G. Nasal drug delivery devices: characteristics and performance in a clinical perspective—a review. Drug Deliv. and Transl. Res. (2013) 3:42-62.
124. Barrett, Song et al., The amyloid precursor protein has a flexible transmembrane domain and binds cholesterol., Science, 336, 2012 Jun. 1; 336(6085):1168-71.
125. Kanekiyo T, Xu H, Bu G, ApoE and Aβ in Alzheimer's disease: accidental encounters or partners? Neuron. 2014 Feb. 19; 81(4): 740-754.
126. Phillips M C, Apolipoprotein E isoforms and lipoprotein metabolism. IUBMB Life. 2014 September; 66(9):616-23.
127. Rapaport, Rao and Igarashi, 2007, Prostaglandins, Leukotrienes and Essential Fatty Acids 77:251-261.
128. Umhau J. C., Zhou W., Carson R. E., et al. Imaging incorporation of circulating docosahexaenoic acid into the human brain using positron emission tomography J. Lipid Res., 50 (2009), pp. 1259-1268.
129. Vitali C, Wellington C L, Calabresi L. HDL and cholesterol handling in the brain. Cardiovasc Res. 2014 Aug. 1; 103(3):405-13.
130. Demeester N, et al., Characterization and functional studies of lipoproteins, lipid transfer proteins, and lecithin:cholesterol acyltransferase in CSF of normal individuals and patients with Alzheimer's disease. J Lipid Res. 2000 June; 41(6):963-74.
131. Vuletic S, et al., Widespread distribution of PLTP in human CNS: evidence for PLTP synthesis by glia and neurons, and increased levels in Alzheimer's disease. J Lipid Res. 2003 June; 44(6):1113-23.

Various references are cited herein, the contents of which are hereby incorporated by reference in their entireties.

What is claimed is:

1. A method of treating a subject in need thereof, comprising administering a therapeutically effective amount of an inhibitor to block or inhibit an interaction between amyloid β (Aβ) and a neuronal lipid.

2. The method of claim 1, where the neuronal lipid is docosahexaenoic acid (DHA) and/or eicosapentaenoic acid (EPA).

3. The method of claim 1, where the subject suffers from mild cognitive impairment or Alzheimer's disease.

4. The method of claim 1, where the subject is a patient with high Aβ load by PET imaging.

5. The method of claim 1, where the subject suffers from Down syndrome and associated cognitive disorders.

6. A method of treating a subject in need thereof, comprising administering a therapeutically effective amount of an inhibitor to block or inhibit an interaction between α-synuclein (αS) and a neuronal lipid.

7. The method of claim 6, where the subject suffers from Parkinson's Disease.

8. The method of claim 7, where the subject suffers from a synucleopathy including dementia with Lewy bodies and multiple system atrophy.

9. A method of blocking or inhibiting an interaction between DHA and Aβ, DHA-CE and Aβ, and/or ApoE and Aβ in a subject in need of such treatment, comprising the use of an inhibitor.

10. The method of claim 9, where the inhibitor is selected from the group consisting of a small molecule, an immunotherapeutic, a soluble Aβ:DHA-CE complex mimetic, a peptidomimetic, and a nanoparticle.

11. The method of claim 10, where said immunotherapeutic is selected from the group consisting of an antibody, a single chain antibody and an antibody fragment.

12. The method of claim 11, where the immunotherapeutic is specific for the Aβ:DHA-CE complex.

13. An assay for identifying an effective blocker or inhibitor of the Aβ:lipid interaction, comprising the steps of:
(i) binding Aβ protein to reacti-bind plates;
(ii) exposing Aβ protein to detectably labeled lipid;
(iii) washing away non-bound lipid;
(iv) exposing Aβ:detectably labeled lipid complex to an inhibitor; and
(v) detecting signal, where the bound lipid (bound to Aβ) is proportional to the detectable signal,
where disruption of Aβ: lipid interaction by the inhibitor results in a decrease in the detectable signal.

14. The assay of claim 13, where said Aβ protein is a form of soluble monomer, oligomer or fibril preparation.

15. The assay of claim 13, where said lipid is docosahexaenoic acid (DHA) or eicosapentaenoic acid (EPA).

16. The assay of claim 13, where the detectable signal is fluorescence.

17. The assay of claim 13, where the decrease on the detectable signal depends on efficacy and affinity.

18. The assay of claim 13, where the inhibitor is at least one of small molecules, immunotherapeutics, soluble Aβ:DHA-CE complex mimetics, peptidomimetics, or nanoparticles.

19. An assay for identifying an effective blocker or inhibitor of the Aβ:lipid interaction, comprising the steps of:
(i) binding lipid to reacti-bind plates;
(ii) exposing lipid to detectably labeled Aβ;
(iii) washing away non-bound Aβ;
(iv) exposing lipid: detectably labeled Aβ complex to an inhibitor; and
(v) detecting signal, where the bound Aβ (bound to lipid) is proportional to the detectable signal,
where disruption of Aβ: lipid interaction by the inhibitor results in a decrease in the detectable signal.

20. An assay for identifying an effective blocker or inhibitor of the Aβ:ApoE interaction, comprising the steps of:
(i) binding ApoE to reacti-bind plates;
(ii) exposing lipid to detectably labeled Aβ;
(iii) washing away non-bound Aβ;
(iv) exposing ApoE: detectably labeled Aβ complex to an inhibitor; and
(v) detecting signal, where the bound Aβ (bound to ApoE) is proportional to the detectable signal,
where disruption of Aβ: ApoE interaction by the inhibitor results in a decrease in the detectable signal.

21. A method of treating a subject in need thereof, comprising administering intranasally a therapeutically effective amount of one or more lipid, wherein said subject suffers from Alzheimer's disease (AD), Down syndrome, Parkinson's disease (PD), a synucleinopathy, dementia with Lewy bodies, or multiple system atrophy.

22. The method of claim 21, where said lipid comprises a dietary polyunsaturated fatty acid, including DHA, EPA, or combinations thereof.

23. The method of claim 21, where said lipid comprises one or more of DHA, EPA, a triglyceride, a phospholipid, a plasmalogen, a cholesterol ester, a ganglioside, and/or a cerebroside.

24. A pharmaceutical composition for treating a subject in need thereof, comprising a therapeutically effective amount of one or more lipid, where said composition is administered to a subject intranasally to promote central nervous system health.

25. The pharmaceutical composition of claim 24, where said lipid is a polyunsaturated fatty acid.

26. The pharmaceutical composition of claim 24, further comprising lipid-based nanoparticles, lipoproteins, lipid emulsions, multifunctional liposomes or gene therapy-based alteration of lipid metabolism and distribution, including ApoE or DHA modifying enzymes including lipid transfer proteins, CETP, LCAT, or other components of reverse cholesterol transport or brain cholesterol metabolism.

27. The pharmaceutical composition of claim 25, where said lipid is selected from the group consisting of DHA, EPA, and a combination thereof.

28. The pharmaceutical composition of claim 24, for use in treating a condition selected from one or more of neurodegeneration, cognitive impairment, Alzheimer's disease (AD), Down syndrome, Parkinson's disease (PD), synucleinopathy, dementia with Lewy bodies and multiple system atrophy.

29. A device for delivering a pharmaceutical composition intranasally, said device comprising a therapeutically effective amount of one or more lipid.

30. The device of claim 29, where said lipid is a polyunsaturated fatty acid.

31. The device of claim 29, further comprising lipid-based nanoparticles, lipoproteins, lipid emulsions, multifunctional liposomes or gene therapy-based alteration of lipid metabolism and distribution, including ApoE or DHA modifying enzymes including lipid transfer proteins, CETP, LCAT, or other components of reverse cholesterol transport or brain cholesterol metabolism.

32. The device of claim 30, where said lipid is selected from the group consisting of DHA, EPA, and a combination thereof.

33. The device of claim 29, for use in treating a condition selected from one or more of neurodegeneration, cognitive impairment, Alzheimer's disease (AD), Down syndrome, Parkinson's disease (PD), synucleinopathy, dementia with Lewy bodies and multiple system atrophy.

34. A method to promote central nervous system health, inhibit neurodegeneration, prevent or treat neurodegenerative disorders, comprising administering a therapeutically effective amount of one or more lipid intranasally, where said neurodegenerative disorder is mild cognitive disorder, Alzheimer's disease, or Down syndrome and associated cognitive disorders, Parkinson's disease (PD) or a synucleinopathy, including dementia with Lewy bodies, and multiple system atrophy.

35. The method of claim 1, where the inhibitor interferes with binding between a lipid and Aβ at SEQ ID NO:1.

36. The method of claim 35, where the inhibitor interferes with binding between a lipid and FFAEDVGSNKGAIIGLMVGGVV (SEQ ID NO:5).

37. The method of claim 6, where the inhibitor interferes with binding between a lipid and αS at SEQ ID NO:2.

38. The method of claim 35, where the inhibitor interferes with binding between a lipid and GAVVTGVT (SEQ ID NO:6).
